(19) United States
(12) Patent Application Publication
(10) Pub. No.: US 2022/0404265 A1
(43) Pub. Date: Dec. 22, 2022

(54) METHOD FOR DETERMINING A PHOTOPROTECTIVE PERFORMANCE VALUE OF A PACKAGE SYSTEM

(71) Applicant: THE CHEMOURS COMPANY FC, LLC, WILMINGTON, DE (US)

(72) Inventors: CHERYL MARIE STANCIK, KENNETT SQUARE, PA (US); PETER JERNAKOFF, WILMINGTON, DE (US); TODD ROBERT EATON, WALLINGFORD, PA (US); ANDRE JOHN BREWER, GREENVILLE, DE (US)

(73) Assignee: THE CHEMOURS COMPANY FC, LLC, WILMINGTON, DE (US)

(21) Appl. No.: 17/777,840

(22) PCT Filed: Nov. 19, 2020
(86) PCT No.: PCT/US2020/061372

§ 371 Date: May 18, 2022

Related U.S. Application Data
(60) Provisional application No. 62/937,441, filed on Nov. 19, 2019.

(51) Int. Cl. G01N 17/00

(52) U.S. Cl. CPC G01N 17/004; G01N 17/002

(57) ABSTRACT

The invention provides novel methods to place a complete package system filled with a sample into a light exposure chamber as a complete unit with periodic monitoring of the contents of the packaging system. Through this approach, it can be determined how the components of a complete package system perform collectively to influence its photoprotective performance enabling a first ever method to quantitate these impacts. In turn this will allow for package designs to be optimized for the photoprotection performances, for the impacts of packaging production defects to be explored, and for the influence of packaging form (e.g., surface area to volume ratio) to be determined. Such capabilities allow a first ever means to optimize package systems for photoprotective performance.

### FIELD OF THE INVENTION

The present invention relates to light protection of packaged goods. Particularly, the invention relates to methods and devices for determining a photoprotective performance value of a complete package system.

### BACKGROUND OF THE INVENTION

Evaluating the light protection performance of packages for consumer-packaged goods applications such as packaged food, beverages and personal care consumer products is of essential need as described, for example, in commonly owned WO 2013/163421, the subject matter of which is herein incorporated by reference.

As described in WO 2013/163421 and commonly owned WO 2013/162947, the subject matter of which is herein incorporated by reference, methods and devices to evaluate the light protection performances of packaging materials are described. Using these methods and devices, the performances of individual packaging materials comprising the components of a package can be assessed to ensure they meet the light protection performance requirements. While it is desirable to have the light protection performance of packaging components that comprise a complete package be similar as discussed, for example in commonly owned WO 2018/183826, it may not be practical to match the performances of the light protection components. In this circumstance, it is useful to understand how the overall light protection performance of a package is influenced by the performances of the various components comprising the package.

For example, a package system may be comprised of a container and corresponding closure. For example, a package system may comprise a bottle, a layer or wrap over a portion of the bottle, a print over a portion of this wrap, and a bottle closure. In this scenario, different light protection performances could be measured for each of these components. However, the teachings of WO 2013/163421 and WO 2013/162947, do not describe the composite impact of different components on the performance of the complete package. Thus, a package designer cannot predict the light protection performance of a final package based upon the performances of the individual components comprising the package.

Generally, it is understood that the performance of the overall package is related to the performances of the individual components; however, this relationship has not been determined nor reported. More so, there may be several factors that would influence this relationship such as the surface area of the various materials presented to light, their uniformity, including the presence of defects in the package constructs, and the surface area to volume ratio of the packaging. Thus, it has been determined that the teachings of WO 2013/163421 are insufficient alone to achieve this goal and that further methods were needed.

Furthermore, a packaging material may be homogenous such that a sample of the packaging material is representative of the photoprotective performance of a larger package fabricated from the material; however, there are several package formats where variation in the material occurs throughout the package construct. For example, in a molded plastic packaging article, the thickness of the wall of the resultant package will differ based upon the features in the mold and thus different performances may result due this inhomogeneity. In another view, a package may be inhomogeneous by design. Examples of inhomogeneous packages include a package where a label only covers a portion of the package surface, a container with a closure that is comprised of a different material and may be a different color, or a package that has printing directly on the package surface that does not uniformly cover the package surface. For this reason, it is useful to determine a method that will allow for evaluation of a complete package that accounts for the differences that may be present throughout the package. Sometimes these differences are evident but sometimes it is difficult to know such differences are present. For example, unanticipated defects may occur in package fabrication resulting in inhomogeneity of the package in thickness, composition, or both. The means to test a package for performance to assess for such inhomogeneity is provided using the teachings of the present invention.

The teachings of WO 2013/163421 and WO 2013/162947 demonstrate how it is beneficial and important to use a light exposure device that provides an accelerated light exposure by use of an intense light source while simultaneously controlling the environment of the light exposure environment. While this is demonstrated for a package component, devices and methods to accomplish light exposures studies of complete package systems is not taught nor envisioned.

The need for devices to provide accelerated light exposures to package systems has been disclosed (https://www.atlas-mts.com/products/standard-instruments/xenon-weathering/suntest/xls), however the designs provide deficiencies that are not readily overcome. The systems lack the means to provide an intense lighted environment at isothermal refrigeration temperatures. Because the Xenon arc light source employed in this disclosed approach itself generates heat, there are not practical means to retrofit such a device with standard cooling designs which in turn limits the minimum disclosed operating temperature to 45° C. Further, at 45° C., the temperature is an order of magnitude higher than retail refrigeration temperature which is often set at 4° C. While temperatures of 45° C. may be useful study temperatures for some applications, for other applications these high temperatures will cause issues in conducting an effective study.

Certain products of interest may change phase or may degrade under such thermal environments beyond their target storage temperature. For example, high temperatures may cause spoilage of food systems. The limitation of using elevated temperature on shelf life studies for certain products is discussed in Sensory Shelf Life Estimation of Food Products, Hough, Chapter 7 Accelerated Storage (CRC Press, 2010).

The light source of a useful device for providing an accelerated light exposure must match the spectrum of the light environment of interest, such as retail light exposure. The aforementioned light chamber (https://www.atlas-mts.com/products/standard-instruments/xenon-weathering/suntest/xls) is relevant to solar spectrums, however, for many packaging applications, the design need is for artificial, indoor lighting conditions such as supermarket or warehouse lighting environments where products may be stored for long times and for retailing. These lighting environments will thus not be well replicated by the spectrum that simulates solar light exposure. Light sources that simulate retail like environments, such as incandescent, fluorescent and light emitting diode (LED), may be useful for light exposure chambers. These light sources however are not found in light exposure chambers for accelerated and controlled studies.

### SUMMARY OF THE INVENTION

The present inventions provide new methods used in conjunction with new devices that provide the ability to assess the performance of a complete package system. In this approach, the ability to produce results both in an accelerated time frame under well controlled and known conditions is achieved.

The invention provides novel methods to place a complete package system filled with a sample into a novel light exposure chamber device as a complete unit with periodic monitoring of the contents of the packaging system. Through this approach, it can be determined how the components of a complete package system perform collectively to influence its photoprotective performance enabling a first ever method to quantitate these impacts. In turn this will allow for package designs to be optimized for the photoprotection performances, for the impacts of packaging production defects to be explored, and for the influence of packaging form (e.g., surface area to volume ratio) to be determined. Such capabilities allow a first ever means to optimize package systems for photoprotective performance in an accelerated and quantitative fashion.

According to an aspect of the invention, a sample can be placed inside a package system, the package system can be equilibrated to a desired control temperature and placed inside a light exposure chamber device, the light exposure chamber device comprising at least on light source, and monitored for change after the package system is exposed to light from the light source while being maintained at the control temperature.

The sample can be a known or unknown entity. In an aspect of the invention the sample can comprise a consumer goods product. For example, the sample can be a solution comprised of a light sensitive constituent of a consumer goods product. For example, the sample can be a solution of aqueous riboflavin. A sample could be a full fluid product, such as a juice, milk, or oil. A sample could be a solution or suspension, or it could be in another form such as a pellet, powder, sheet, or other form. A sample could be a solid, liquid, or other form.

The sample can be monitored for change by removing the sample from the light exposure chamber after a defined duration of light exposure, and by removing a closure of the package system, and by placing a probe or monitor into the package for an evaluation. An aliquot of the sample could be removed from the package system after a defined duration of light exposure at the control temperature. A probe could be mounted into the package system for continual monitoring of the sample.

Package systems could be monitored by removing them from the light exposure chamber and assessing their contents. Package systems could also be monitored in situ with monitoring devices that measure the sample within the package system while they are in the light exposure chamber.

According to an aspect of the invention, a device is provided that can provide light exposure to a package system. The device includes a contained enclosure to isolate an exposure environment with the means to control a temperature within the enclosure, at least one light source of controlled spectral intensity selected from the group consisting of LED, fluorescent, and halogen, the light source being capable of simulating light used in retail but at a defined and known intensity; at least one positioning mechanism located in the enclosure for defining the placement of one or more package systems within the enclosure relative to the at least one light source to ensure controlled light delivery to the package system; and one or more monitoring instruments used with the device to confirm at least one stability measure of the light exposure conditions within the enclosure during light exposure evaluations.

### BRIEF DESCRIPTION OF THE FIGURES

FIG. 1 is a schematic drawing of a light exposure device according to an aspect of the invention.

FIG. 2 is a schematic drawing detailing the inner portion of a light exposure device according to an aspect of the invention.

FIG. 3 is a schematic drawing demonstrating one embodiment of a light exposure device according to the present invention.

FIG. 4 is a schematic drawing detailing a further embodiment of a light exposure device according to the present invention.

FIG. 5 is a schematic drawing detailing a further embodiment of a light exposure device according to the present invention.

FIG. 6 is a schematic drawing detailing a further embodiment of a light exposure device according to the present invention.

FIG. 7 is a schematic drawing detailing a further embodiment of a light exposure device according to the present invention.

FIG. 8 is a schematic drawing detailing a still further embodiment of a light exposure device according to the present invention.

FIG. 9 is a schematic drawing on an assembly of a packaging system with septum according to an aspect of the invention.

FIG. 10 is a schematic drawing of the sequential steps of preparing an assembly of a foil control system with septum according to an aspect of the invention.

FIG. 11 is a graph showing riboflavin concentration versus light exposure time obtained in Example 1.

FIG. 12 is a graph showing the natural log of riboflavin concentration versus light exposure time obtained in Example 1.

FIG. 13 is a graph showing the natural log of riboflavin concentration versus light exposure time with linear fits obtained in Example 1.

FIG. 14 is a schematic drawing detailing an assembly of a packaging system with cap according to an aspect of the invention.

FIG. 15 is a schematic drawing detailing an assembly of a packaging system with cap and foil sleeve according to an aspect of the invention.

FIG. 16 is a graph showing the natural log of riboflavin concentration versus light exposure time obtained in Example 2.

FIG. 17 is a schematic drawing detailing an assembly of a foil packaging system with cap according to an aspect of the invention.

FIG. 18 is a schematic drawing of a assembly of a foil covered packaging system with controlled defects according to an aspect of the invention.

FIG. 19 is a graph showing the natural log of riboflavin concentration versus light exposure time obtained in Example 3.

FIG. 20 is a graph showing the chlorophyll content and correct sensory response of packaged olive oil as a function of light exposure time obtained in Example 4.

FIG. 21 is a graph showing the observed pseudo first order rate constants for riboflavin decline in light exposed packaged milk for a set of package systems obtained in Example 5.

FIG. 22 is a graph showing the observed pseudo first order rate constants for riboflavin decline in light exposed packaged milk compared to light exposed RF solution for a set of package systems obtained in Example 5.

### DETAILED DESCRIPTION OF THE INVENTION

The following definitions and abbreviations are to be use for the interpretation of the claims and the specification.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “consists of,” or variations such as “consist of” or “consisting of,” as used throughout the specification and claims, indicate the inclusion of any recited integer or group of integers, but that no additional integer or group of integers may be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether modified or not by the term “about”, the claims include equivalents to the quantities. In one embodiment, the term “about” means within 10% of the reported numerical value, preferably within 5% of the reported numerical value.

The invention provides methods for determining a photoprotective performance value of a package system, the method comprising:
(a) providing a light exposure chamber;
(b) providing a package system containing one or more samples to form a filled package system;
(c) providing at least one light source;
(d) equilibrating the filled package system to a desired control temperature;
(e) positioning the filled package system inside the light exposure chamber at a desired distance from the at least one light source;
(f) exposing the filled package system to the at least one light source for one or more durations while maintaining the desired control temperature;
(g) measuring changes to the sample at the one or more durations to generate one or more data points;
(h) using the one or more data points to determine the photoprotective performance value of the package system.

The invention further provides a device for providing light exposure to a package system comprising: (a) a contained enclosure to isolate an exposure environment; (b) means to control a temperature within the enclosure; (c) at least one light source of controlled spectral intensity selected from the group consisting of LED, fluorescent, and halogen; (d) at least one positioning mechanism located in the enclosure for defining a package system location within the enclosure relative to the at least one light source to ensure controlled light delivery; and (e) at least one monitoring instrument to confirm an at least one stability measure of the light exposure conditions within the enclosure during light exposure evaluations.

In an aspect of the invention the at least one monitoring instrument monitors at least one of light intensity, temperature, and humidity.

In a further aspect of the invention the temperature within the enclosure is controlled at a temperature of from about 1° C. to about 25° C.

In a further aspect of the invention the temperature within the enclosure is controlled at a temperature of from about 25° C. to about 40° C.

In a further aspect of the invention the temperature within the enclosure is controlled at a temperature of above about 40° C.

In a still further aspect of the invention the at least one positioning mechanism is rotatable.

Positioning of a complete packaging system within a light exposure chamber device must allow for uniform exposure of three-dimensional packaging systems of varying shape and size to allow for a robust accelerated light exposure. In the device of the present invention, the chamber has light directed to every surface of the package. To further ensure uniform light dose is received to the package surfaces, the packages themselves are rotated within the chamber to provide a sampling of the lighted environment and to ensure that each package in the chamber receives identical light dose. By ensuring the consistency of the light dose to the multiple packages in the chamber, robust and direct comparisons can be made between packages studied within the chamber. Further, the delivery of an accurate and consistent light dose achieved with the light exposure device of the present invention allows for performance data from subsequently exposed packages to be directly and quantitatively compared to light exposure experiments conducted at earlier times. Finally, actively collecting and monitoring light exposure dose and temperature data concurrent with exposure provides a confirmation of the experimental conditions.

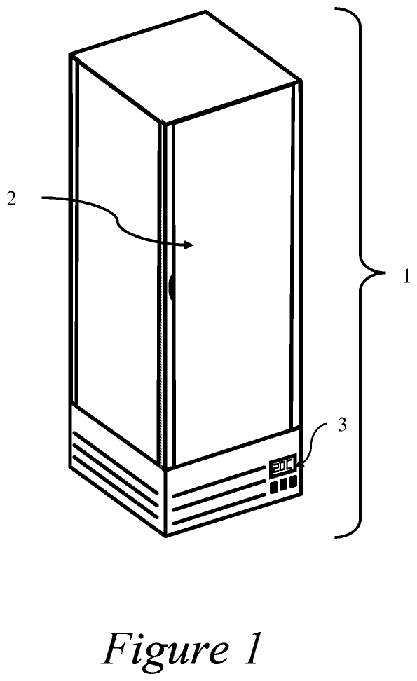

Many aspects of the presentation of a light exposure to package system have been conceived in various embodiments of this invention as shown in FIGS. 1-7:

### FIG. 1: Light Exposure Device

FIG. 1 is a schematic drawing of a preferred embodiment that provides a device for providing light exposure to one or more package systems. The device includes light exposure chamber 1, which includes door 2 and a means for maintaining a desired temperature of the contents of a package system, the means for maintaining a desired temperature includes control panel 3.

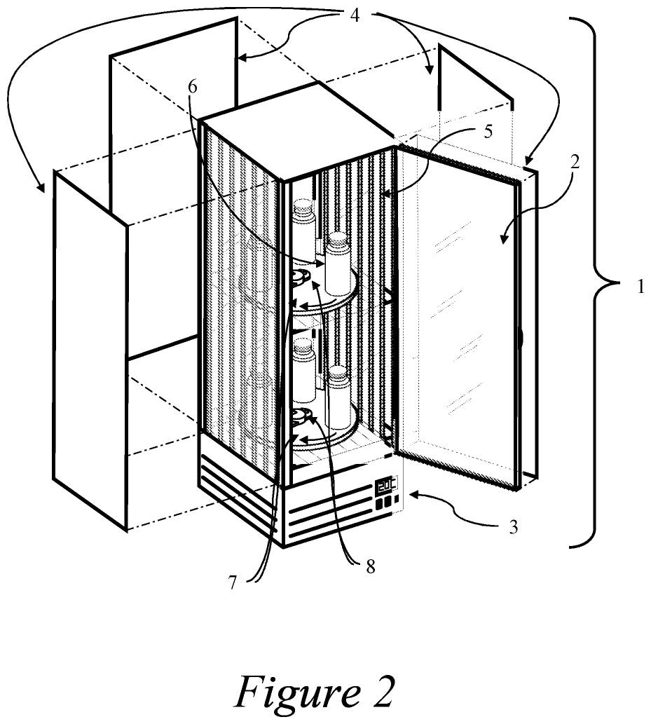

FIG. 2: Light Exposure Device with Package Systems

FIG. 2 shows the interior elements of the light exposure chamber of FIG. 1. Light exposure chamber 1 comprises door 2, reflective sheeting 4, means for maintain a desired temperature 3, multiple light sources 5, which are arranged to substantially surround package systems 6. FIG. 2 also includes multiple rotating members 7. In an aspect of the invention, multiple package systems 6 can be filled package systems with closures as shown. In operation, the filled package systems 6 are positioned on the multiple rotating members 7 at an established control temperature. Door 2 is closed and multiple light sources 5 are illuminated, while rotating members 7 are rotated about their axis at a desired rpm value, while the filled package systems 6 are held at the desired control temperature for one or more durations. The device environment is monitored using sensors 8 to measure the light intensity and temperature proximate to the package systems 6 concurrent with light exposure. The contents of the filled package systems can be measured for changes after each duration to determine any change to the contents. The changes to the contents serve as data points which can be used to determine the photoprotective performance value of the package system.

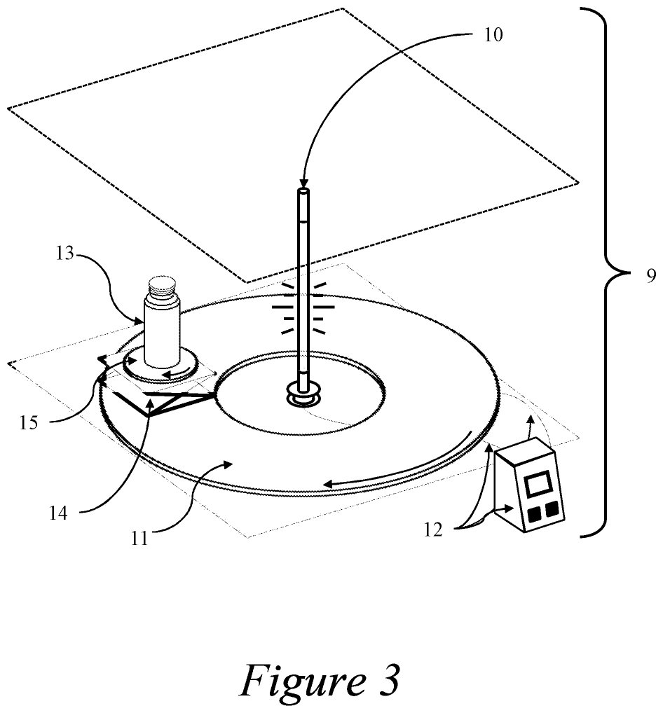

### FIG. 3: Light Exposure Chamber, Central Light Source

FIG. 3 is a schematic drawing of yet another alternative embodiment of the invention. FIG. 3 shows the interior view of a light exposure chamber 9 with centrally located light source 10, located at the axis of rotatable member 11. Temperature, light and rotation control means 12 is also shown. The light exposure chamber 9 includes package system 13 (which can be a filled package system including a sample and corresponding closure, as shown) arranged on top of package system holder 14, which includes a second rotatable member 15. Second rotatable member 15 provides a means to rotate the package system 13 about its own axis, while the package system 13 is rotated about the light source 10. System holder 14 can adjust the sample vertical position above the rotatable member 11 for fine-tuned light adjustments.

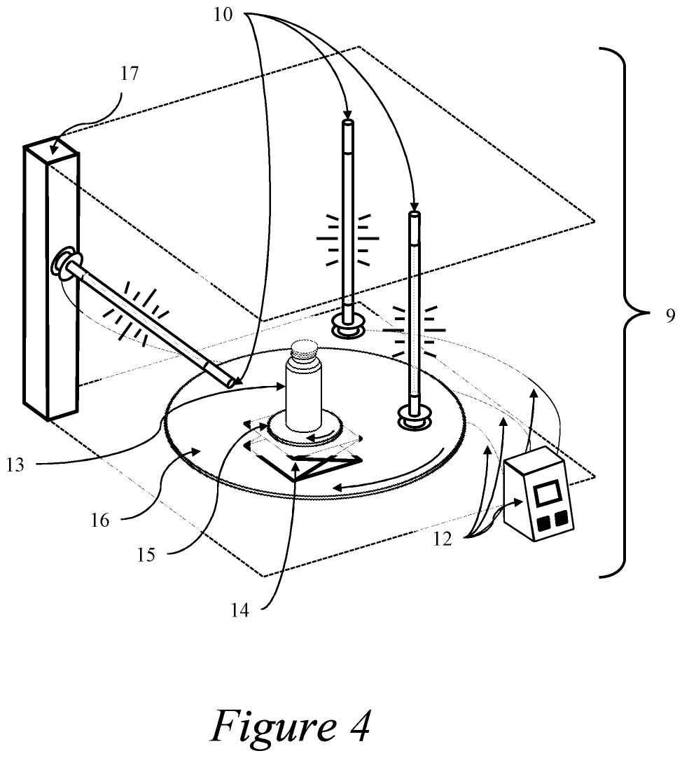

### FIG. 4: Light Exposure Chamber and Package System Positioning

FIG. 4 is a schematic drawing of yet another alternative embodiment of the invention. FIG. 4 shows the interior of a light exposure chamber 9 with temperature, light and rotation control means 12, rotatable member 16 and second rotatable member 15, with package system 13 positioned on rotatable member 15, which is located on package system holder 14. Multiple light sources 10 are provided and one is located at a fixed position on rotatable member 16, at a predetermined, fixed distance from rotatable member 16 and extending vertically 17 with relation to rotatable member 16 and 15, and also at a predetermined, fixed distance from rotatable member 16 and extending horizontally with relation to rotatable member 15. System holder 14 can adjust the sample vertical position above the rotatable member 16 for fine-tuned light adjustments.

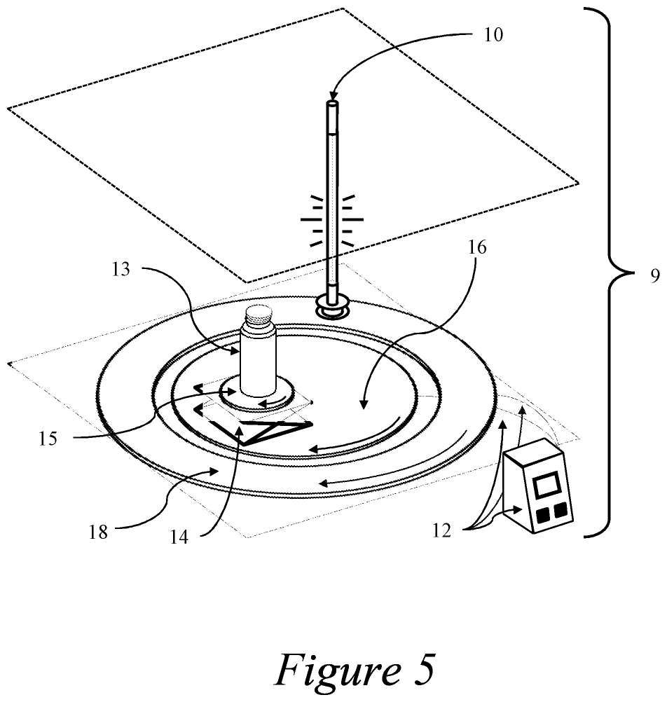

### FIG. 5: Light Exposure Chamber, Package System and Light Source Positioning

FIG. 5 is a schematic drawing of yet another alternative embodiment of the invention. FIG. 5 shows the interior of a light exposure chamber 9 with light source 10, temperature, light and rotation control means 12, rotatable member 18 and second rotatable member 16, with package system 13 positioned on rotatable member 15, which is located on package system holder 14. Rotatable member 18, in conjunction with rotatable member 16 creates various periodic lighting scenarios as the light source 10 and packaging system 13 rotate independently or dependently.

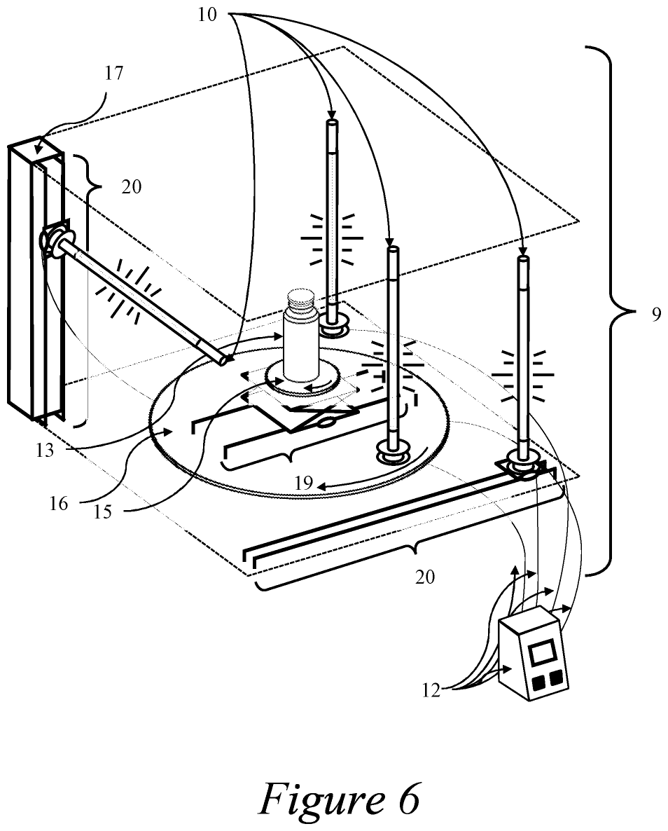

### FIG. 6: Light Exposure Chamber, Package System Positioning and Varied Light Sources

FIG. 6 is a schematic drawing of yet another alternative embodiment of the invention. FIG. 6 shows the interior of light exposure chamber 9 with temperature, light and rotation control means 12, rotatable member 16, second rotatable member 15, located on package system holder system 19, which is movable in a translation across the surface of rotatable member 16 and provides vertical sample position adjustment above the rotatable member 16 for fine-tuned light adjustments in conjunction with lateral adjustment. The light exposure chamber also includes multiple light sources 10, which are located at a fixed positions on rotatable member 16, at a predetermined, fixed distances from rotatable member 16, and with two light sources located a predetermined distance from rotatable member 16 and being movable, in one case in a back-and-forth direction horizontally from rotatable member 16, and in the other case in a back-and-forth direction vertically from rotatable member 16 using lateral stages 20.

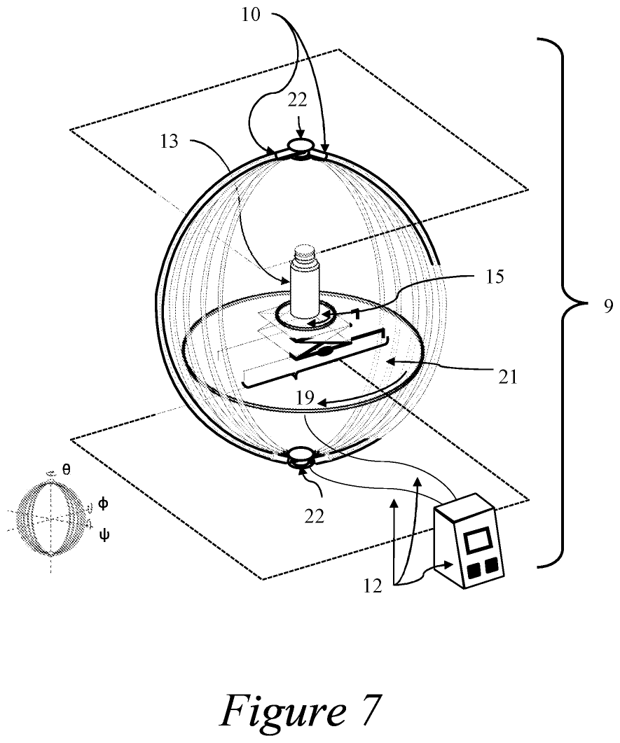

### FIG. 7: Light Exposure Chamber and Package System Positioning, Spherical

FIG. 7 is a schematic drawing of yet another alternative embodiment of the invention. FIG. 7 shows the interior of light exposure chamber 9 with temperature, light and rotation control means 12, rotatable member 21 (similar to previous rotatable member 16 but made of a translucent material), second rotatable member 15, located on package system holder system 19, which is movable across the surface of rotatable member 21. The light exposure chamber also includes multiple light sources 10, which substantially surround package system 13 with support and connective members 22. The entire light source assembly can rotate on any axis.

### Light Exposure Devices and Chambers:

Light exposure chamber devices can be of any suitable shape and include at least one light source located therein and at least one means to maintain the filled package system at a desired control temperature.

A light exposure chamber that allows for active control of the exposure conditions is preferred. The chamber must include means for controlling light intensity. The chamber light intensity and temperature are monitored to ensure consistency.

Preferred light exposure chamber devices provide means to control the temperature within the chamber. The temperature within the chamber can be controlled at or about 1° C., 2° C., 3° C., 4° C., 5° C., 6° C., 7° C., 8° C., 9° C., 10° C., 15° C., 20° C., 22° C., 25° C., 30° C., 37° C., 40° C., 50° C., 55° C., or 60° C. The chamber can be insulated to maintain the desired temperature control.

Within the light exposure chamber, means to mount package systems in a controlled fashion can be provided. As the intensity of light reaching a package system will depend upon the distance and orientation to the light sources, package systems should be at defined distances and positions as oriented from the light source.

Exemplary embodiments of suitable light exposure devices are shown in the Figures and discussed herein below.

### Package Systems:

Package systems can comprise any suitable material or multiple materials and include a container portion and in an aspect of the invention can further comprise at least one opening or closure portion. The container opening or closure can be the same or different material. The container, opening, and closure can be any suitable size and/or shape. In an aspect of the invention the package system container comprises a wall thickness of from about 1 mil to about 100 mil. The materials may be of rigid or flexible construction or combination thereof. The package system may comprise and inerting gas, fluid, or other absorbent material to inert, partition, release, or sequester material as part of the function of the package. This may be part of the packaging system.

### Light Source:

The light source can be any suitable light source to produce the desired light intensity, stability, and spectral characteristics. Depending upon the needs of the experiment, light sources employed may include incandescent light sources, fluorescent light sources, arc discharge lamps, LEDs (light emitting diodes), and/or laser light sources. For example, these light sources include but are not limited to carbon arc, mercury vapor, xenon arc, tungsten filament, or halogen bulbs. In one embodiment, the light source is a xenon arc lamp. In another embodiment the light source is LED. In another embodiment, there are multiple light sources of one or more types. The light source can be powered by a battery. The light source may be powered by standard electricity.

In certain embodiments, the light source can provide an intensity of between about 0.001 W/cm² and about 5 W/cm² as measured at the defined monitoring position. In other embodiments, the light source is capable of providing an intensity of at least about 0.001 W/cm², 0.005 W/cm², 0.007 W/cm², 0.01 W/cm², 0.05 W/cm², 0.1 W/cm², 1 W/cm², 2.5 W/cm², or 5 W/cm² as measured at the defined monitoring position. In further embodiments, the light source can provide an intensity of not more than about 0.001 W/cm², 0.005 W/cm², 0.007 W/cm², 0.01 W/cm², 0.05 W/cm², 0.1 W/cm², 1 W/cm², 2.5 W/cm², or 5 W/cm² as measured at the defined monitoring position. In further embodiments, the light source can provide an intensity between about 0.005 W/cm² and about 4 W/cm², between about 0.007 W/cm² and about 3 W/cm², between about 0.01 W/cm² and about 2.5 W/cm², between about 0.05 W/cm² and about 2 W/cm², or between about 0.1 W/cm² and about 1 W/cm² as measured at the defined monitoring position.

In an embodiment the light source intensity is characterized by the units of lux using a light intensity meter with an intensity of 5000 lux, 6000 lux, 7000 lux, 8000 lux, 9000 lux, 10,000 lux, 11,000 lux, 12,000 lux, 13,000 lux, 14,000 lux or 15,000 lux at the defined monitoring position. In further embodiments, the light source can provide an intensity between about 20,000 and about 2000 lux, between about 18,000 and about 2500 lux, between about 10,000 and about 3000 lux, between about 12,000 lux and about 5000 lux, or between about 16,000 lux and about 4000 lux as measured at the defined monitoring position.

In other embodiments, the light source is capable of producing light with a spectral signature of about 200 nm to about 2000 nm. In other embodiments, the light source is capable of providing light at a wavelength of at least about 200 nm, 220 nm, 240 nm, 260 nm, 280 nm, 290 nm, 300 nm, 350 nm, 400 nm, 450 nm, 500 nm, 550 nm, 600 nm, 650 nm, 700 nm, 750 nm, 800, nm, 900 nm, 1000 nm, 1250 nm, 1500 nm, 1750 nm, or 2000 nm. In further embodiments, the light source is capable of providing light at a wavelength of not more than about 200 nm, 220 nm, 240 nm, 260 nm, 280 nm, 290 nm, 300 nm, 350 nm, 400 nm, 450 nm, 500 nm, 550 nm, 600 nm, 650 nm, 700 nm, 750 nm, 800, nm, 900 nm, 1000 nm, 1250 nm, 1500 nm, 1750 nm, or 2000 nm. In still further embodiments, the light source is capable of providing a spectral signature of about 220 nm to about 1750 nm, about 240 to about 1500 nm, about 260 to about 1250 nm, about 290 to about 1000 nm, about 200 to about 400 nm, about 350 to about 750 nm, or above about 750 nm. In additional embodiments, the light source is capable to provide spectral signature including portions of the UV spectrum. In certain embodiments UV spectrums like that present in solar electromagnetic radiation are desired, including UVA (315 to 400 nm) and UVB (280 to 315 nm) components. Such soft UV and intermediate UV light are components of solar electromagnetic radiation.

Any electromagnetic radiation source (LED, Halogen, fluorescent (bio, non-bio, etc.), Luminescent (bio, non-bio, etc.), incandescent, Arc (Xe, carbon, etc.), LASER, MASER, X-ray, Radio, Microwave, the sun, the moon, etc.), either as is or conditioned (filtered (low-pass, high-pass, bandpass, multi-bandpass, etc.), polarized (described via any Jones vector, coherency matrix, Poincare Sphere, etc.), amplified via gain medium, attenuated, etc.) either coherent or incoherent, and in any combination of the above.

In other embodiments a spectral filter or filters can be used in conjunction with any of the aforementioned light sources to provide spectral modification as desired to match the light source to the application of the package system under evaluation.

### Temperature Control and Desired Temperature:

Temperature control can be passive or active. Passive temperature control is achieved in the chamber by use of chamber materials, such as heat absorbing materials, and design, such as the use of insulation and doors to maintain the temperature environment in the chamber to allow for thermal stability. In addition, passive temperature control is achieved by sample observation procedures that limit changes to the chamber temperature. Active temperature control is achieved in the chamber by use of thermostatic heating and cooling devices whereby temperature is measured, and the measurement is used by the chamber thermostat to determine the level of heating or cooling to be delivered to the chamber.

Temperature control to a desired chamber temperature within 5° C. is useful, more preferred is to control temperature within 2° C., even more preferably 1° C. Packages are placed into the chamber and thermal equilibrium of the package in the chamber allows the package content temperature to become equal to the chamber temperature. Controlling temperature at refrigeration temperature, 1° C., 2° C., 3° C., 4° C., 5° C., 6° C., 7° C., 8° C., 9° C., 10° C. is desired for packages that are maintained in cold storage. Maintaining packages at intermediate temperatures is useful for many applications where products are stored or retailed outside, indoors, or in warehouses. In these cases, temperature ranges from the above refrigeration temperatures to ambient ranges are relevant including from 10° C., 15° C., 20° C., 25° C., 30° C., 35° C., to 40° C.

### Samples:

The sample contained within the package system of study can be a known or unknown entity. In an aspect of the invention the sample can comprise a consumer goods product. For example, the sample can be a solution comprised of a light sensitive constituent of a consumer goods product. For example, the sample can be a solution of aqueous riboflavin. A sample could be a full fluid product, such as a juice, milk, or oil. A sample could be a solution, or it could be in another form such as a pellet, powder, sheet, or other form. A sample could be a solid, liquid, or other form or a mixture thereof.

The sample can be monitored for change by removing the packaged sample from the light exposure chamber after a defined duration of light exposure at the desired control temperature and by removing the closure and placing a probe or monitor into the package for an evaluation of the sample within. An aliquot of the sample could be removed from the package after a defined duration of light exposure for evaluation ex situ. A probe could be mounted into the package for continual monitoring of the sample in situ. The sample may be monitored for change while it remains within a closed package.

In particular embodiments, the sample comprises a photosensitive entity. In an aspect of the invention the photosensitive entity is selected from:
i. natural and synthetic food additives, dyes, and pigments (e.g., curcumin, erythrosine);
ii. chlorophyll (all variants);
iii. myoglobin, oxymyoglobin, and other hemeproteins;
iv. water and fat soluble essential nutrients, minerals, and vitamins (e.g., riboflavin, vitamin A, vitamin D);
v. food components containing fatty acids, particularly polyunsaturated fatty acids;
vi. oils (e.g., soybean oil);
vii. proteins (e.g., proteins derived from the amino acids tryptophan, histidine, tyrosine, methionine, cysteine, etc.);
viii. pharmaceutical compounds;
ix. personal care and cosmetic formulation compounds and their components;
x. household chemicals and their components; and
xi. agricultural chemicals and their components.

### Filled Package System:

Filled package systems comprise a package system containing at least one sample. In addition to the at least one sample, the filled package system can include an atmosphere. The atmosphere can comprise an inerting gas or gas mixture including gasses such as air, nitrogen, argon, or carbon dioxide. Other gases used in modified atmosphere package applications can be used. The inerting gas or gas mixture may displace all or part of the air or atmosphere in the package system. The package system may contain an absorbent material integrated into the package construct or added as a discrete construct, such as a pouch of absorbent. The absorbent material may adsorb gas, such as oxygen, or vapors such as water vapor or humidity. Alternatively, the atmosphere of the package can be removed from the package system for a vacuum packaging approach. In both cases where the atmosphere is modified or removed from the package, the evaluation of the contents of the package would be performed accounting for these conditions.

### Evaluating Package Contents Through the Light Exposure Period:

The inventive method does not merely assess for light passing through the package system but rather monitors the consequences of the light received to the sample contained in the package system from the light exposure system. This can be accomplished by monitoring the changes to the sample. These changes then provide information about the overall performance of the package system for light protection.

The changes to the sample can be monitored continuously during the light exposure (e.g., in situ) or discretely by pausing the light exposure for evaluations to monitor the sample by assessing the sample for analysis or by removing an aliquot of the sample for analysis. When aliquots are removed from the package, tests can be performed to understand how the removal of aliquots influences the response of the sample.

According to an aspect of the invention, a sample can be placed inside a package system and monitored for change after the package system is exposed to light at the desired control temperature.

Packages could be monitored by removing them from the light exposure chamber and assessing their contents. Packages could also be monitored in situ with monitoring devices that measure the package contents while they are in the light exposure chamber.

UV-Visible spectroscopy can be used to monitor the changes of a marker sample. Marker samples may be solutions of riboflavin can be monitored using a probe placed into the package to determine the changes to the UV-visible spectra that occur as a function of the light exposure time. Other methods to monitor sample changes may employ chromatography (gas or liquid chromatography), LAB color, or any other analytical methodology employed to study chemicals.

### EXAMPLES

The present invention is further defined in the following Examples. The Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Abbreviations

m=meters
mm=millimeters
μm=micrometers (microns)
nm=nanometers
L=liter
mL=milliliters
g=grams
mg=milligrams
° C.=degrees Celsius
C=degrees Celsius
W=watts

### Example 1

Light exposure was provided to a set of package systems filled with a solution of riboflavin, a photosensitive nutrient species. The riboflavin solution contents of the package systems were measured periodically after defined intervals of light exposure for riboflavin decline and used to monitor the performance of the package system for photoprotective performance.

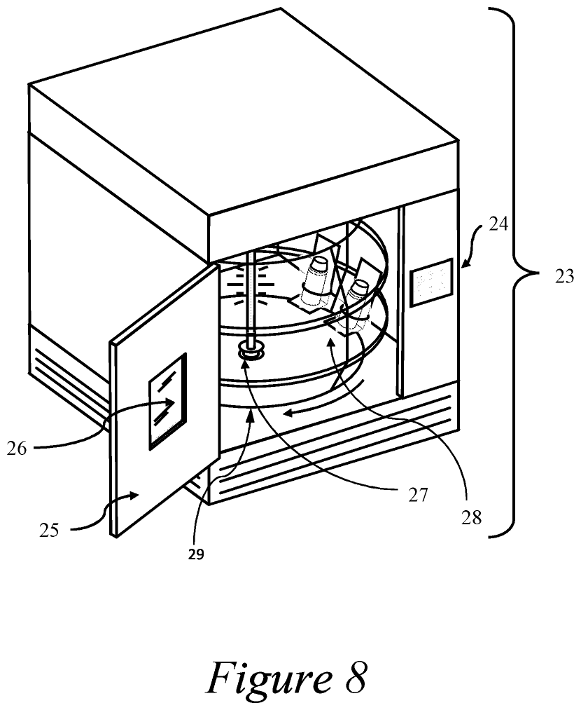

Example 1 may be best understood with reference to FIGS. 8-13:

### FIG. 8: Light Exposure Chamber (Weather-Ometer)

FIG. 8 is a schematic drawing of a preferred embodiment that provides a system for determining the photoprotective performance value of one or more package systems. The system includes light exposure chamber 23, which includes a means for maintaining a desired temperature of the contents of a package system, the means for maintaining a desired temperature includes control panel 24. The light exposure chamber 23 also can include door 25, which can include window 26. As shown, light exposure chamber 23 can include light source 27, which can be centrally located in relation to one or more package systems 28. The light source 27 is located a fixed distance from one or more package systems 28 and is centrally positioned on rotatable member 29. Door 25 is closed and light source 27 is energized to provide a light beam that impinges upon filled package systems 28. Rotatable member is rotatable about its axis and the filled package systems are held at the desired control temperature for one or more durations. The contents of the filled package systems can be measured for changes after each duration to determine any change to the contents. The changes to the contents serve as data points which can be used to determine the photoprotective performance value of the package system.

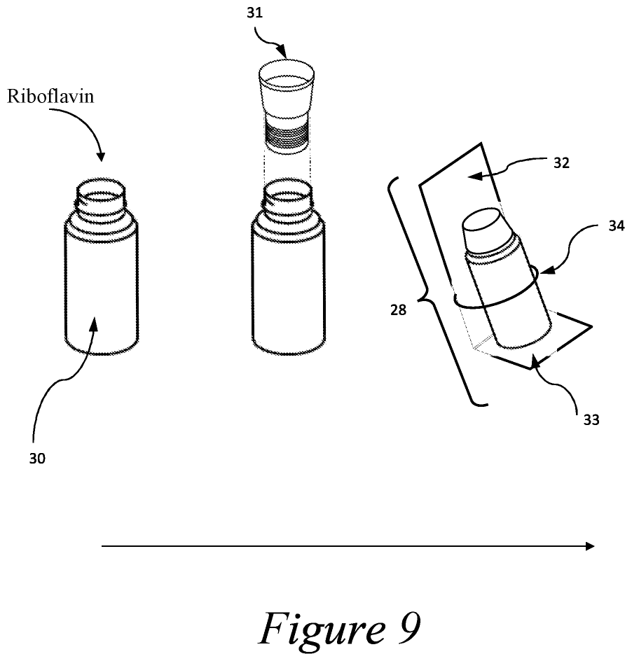

FIG. 9: Assembly of a Packaging System with Septum

According to an aspect of the invention, a package 30 is filled, sealed with a rubber septum 31 and can be placed onto a holder consisting of a L-bracket with backing 32 and bottom shelf 33, with the filled package system equilibrated to a desired control temperature. The package is secured to the backing 32 by means of zip-tie 34. The filled, sealed with septum and secured package is referred to as a package assembly 28.

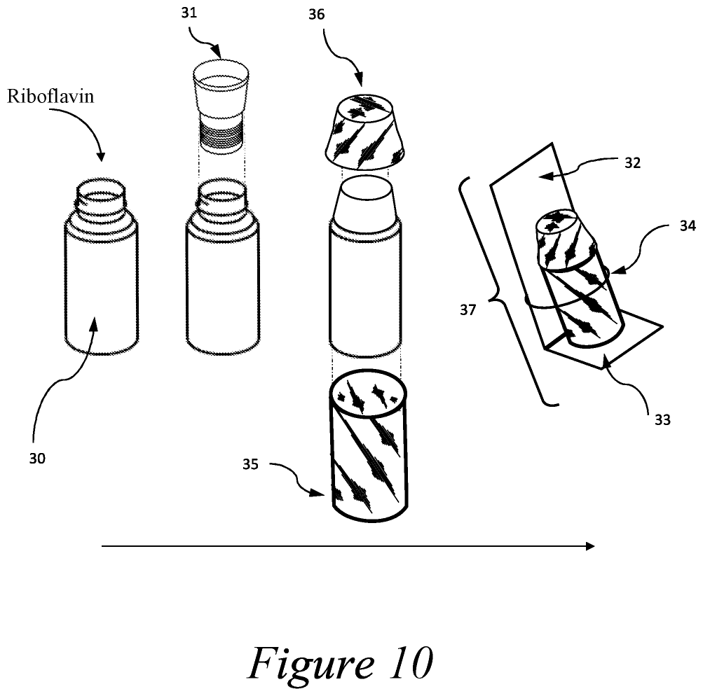

### FIG. 10: Assembly of a Foil Control System

According to an aspect of the invention, a package 30 is filled, sealed with a rubber septum 31 and further covered in foil 35 up to the point where the exterior of the bottle transitioned to the subsequently applied bottle closure (the transfer bead) and further capped with foil top 36 and can be placed onto a holder consisting of a L-bracket with backing 32 and bottom shelf 33, with the filled package system equilibrated to a desired control temperature. The package is secured to the backing 32 by means of zip-tie 34. The filled, sealed and secured Foil control system is referred to as a package assembly 37.

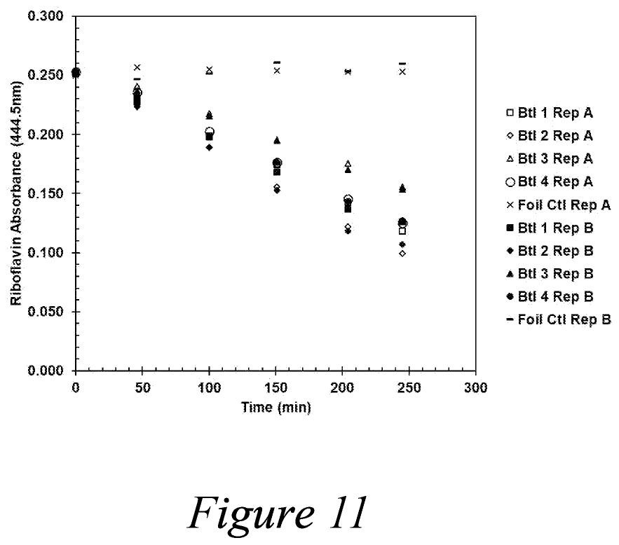

### FIG. 11: Riboflavin Absorbance Versus Light Exposure Time

FIG. 11 shows the decline in riboflavin absorbance for riboflavin solutions contained within package systems Bottle 1 (Btl 1, square symbols), Bottle 2 (Btl 2, diamond symbols), Bottle 3 (Btl 3, triangle symbols), and Bottle 4 (Btl 4, circle symbols) as a function of light exposure time. Open symbol are used for Replication A and closed symbols are used for Replication B. Also shown are data for the foil control package system (x, −) for Replication A and B, respectively.

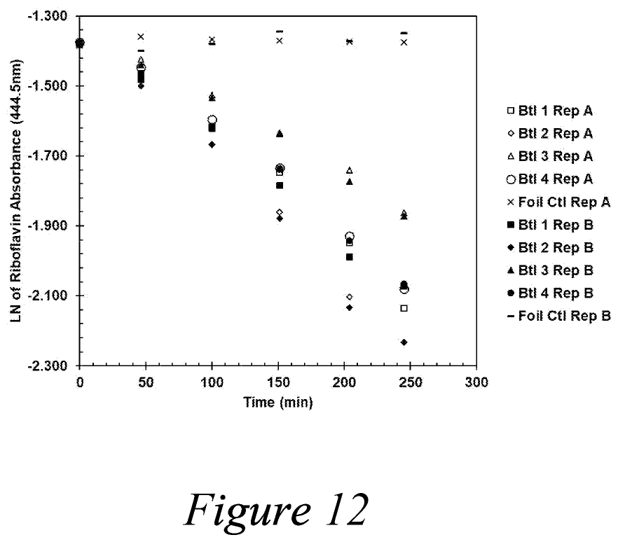

### FIG. 12: Natural Log of Riboflavin Absorbance Versus Light Exposure Time

FIG. 12 shows the decline in the natural logarithm (ln) of riboflavin absorbance for riboflavin solutions contained within package systems Bottle 1 (Btl 1, square symbols), Bottle 2 (Btl 2, diamond symbols), Bottle 3 (Btl 3, triangle symbols), and Bottle 4 (Btl 4, circle symbols) as a function of light exposure time. Open systems are used for Replication A and closed symbols are used for Replication B. Also shown are data for the foil control package system (x, −) for Replication A and B, respectively.

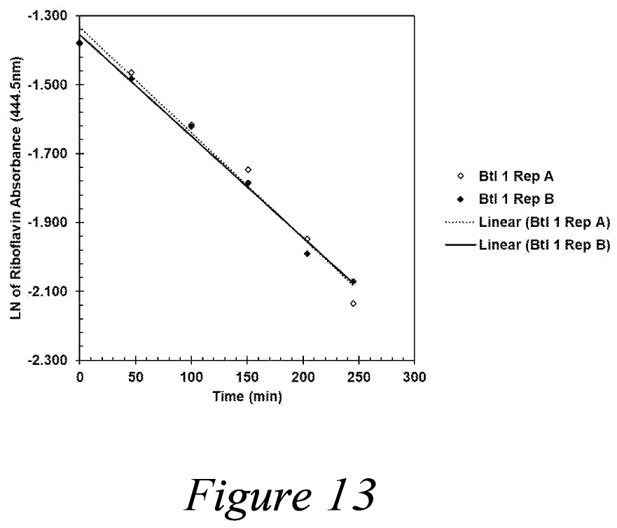

FIG. 13: Natural Log of Riboflavin Absorbance Versus Light Exposure Time with Linear Fits

FIG. 13 shows the decline in the natural logarithm (ln) of riboflavin absorbance for riboflavin solutions contained within package systems Bottle 1 (BtL 1) as a function of light exposure time. Open systems are used for Replication A and closed symbols are used for Replication B. Also shown are data for the linear regression of the data for Replication A (dashed line) and B (solid line).

### Riboflavin Solution:

Aqueous solutions of riboflavin, a light sensitive species, at a 15 mg/L concentration (pH 6.4-6.5) were prepared by first adding ˜300 mL of de-ionized water to a 4-liter glass volumetric flask. Sodium dihydrogen phosphate monohydrate (19.871 g; Sigma-Aldrich, St. Louis, Mo.), sodium monohydrogen phosphate heptahydrate (15.012 g, Sigma-Aldrich), and sodium chloride (0.362 g, Sigma-Aldrich) were then added to the flask and dissolved (approximately one-minute manual agitation). Riboflavin (0.0600 g, Sigma-Aldrich) was then quickly added to the flask followed by another ˜700 mL of de-ionized water. After riboflavin dissolution with approximately two minutes of manual agitation, the flask was quickly filled to volume mark with de-ionized water and a magnetic stir bar was added. The flask was then immediately covered with aluminum foil and the flask contents allowed to stir on magnetic stir plate at room temperature for between 1.5 and 4 hours. The prepared solution was then quickly vacuum filtered using a 0.2-1.0 μm pore size filter membrane/flask apparatus (part number 10040-440; VWR, Radnor, Pa.). Collected filtrate was stored at 4° C. in 1 liter, aluminum foil covered, amber colored storage bottles with threaded closures.

### Riboflavin Absorption Spectrum:

Riboflavin absorption spectra were obtained using a 4 mm wide, transflection, UV-VIS dip probe (Falcata 4; Hellma, Müllheim, Germany) that was manually inserted at periodic intervals into riboflavin solutions for analysis. A tungsten/deuterium ultraviolet-visible-near infrared (UV-VIS-NIR) light source (DH-mini; Ocean Insight, Largo, Fla.) provided light to said probe while return light was processed using a spectrometer (USB2000+, 200-850 nm wavelength range; Ocean Insight) and associated software (SpectraSuite; Ocean Insight). The magnitude of the 444.5 nm absorbance peak of riboflavin was used as a direct proxy for riboflavin concentration.

### Light Exposure Apparatus:

Light exposures were performed with a xenon arc lamp, 27 in FIG. 8, with a weatherometer (Ci5000 Xenon Weather-Ometer®; Atlas Material Testing Solutions, Mount Prospect, Ill.), 23 in FIG. 8. Packaging system studies including both test and control packaging system samples were performed.

During each light exposure event, the following weatherometer settings were utilized: chamber black panel temperature=63° C., chamber air temperature=50° C., chamber relative humidity=uncontrolled (no water spray utilized, ˜5% relative humidity was observed during each light exposure event), xenon arc lamp irradiance at 340 nm=0.35 W/m². Using a handheld optical power meter and associated sensor (1919-R Optical Power Meter and 919P-020-12 Thermopile Sensor; Newport Corporation, Irvine, Calif.), the latter setting was found to result in a broad spectrum (190-11000 nm) light irradiance of about 1300 W/m² at the sample positions located circumferentially along the bottom support ring of the rotatable weatherometer sample carousel, 29 in FIG. 8. During light exposure, the sample carousel rotated (approximately one revolution per minute) about the light source to create a uniform light exposure to the package systems, 28 in FIGS. 8 and 9 and 37 in FIG. 10.

### Calculation of the Pseudo-First Order Rate

### Constant and Half-Life for Riboflavin Degradation:

The decomposition of riboflavin in dilute aqueous solution under an air atmosphere has been shown to follow pseudo-first order rate kinetics when said solution is exposed to ultraviolet or visible light (see Ahmad, I.; Fasihullah, Q.; Noor, A; Ansari, I. A.; Ali, Q. Nawab Manzar, International Journal of Pharmaceutics (2004), 280(1-2), 199-208). More specifically, under conditions during which the energy distribution of light that is incident upon said solution is held constant, said decomposition can be described by the following integrated rate expression:

Ln[Riboflavin]ₜ=(−k′×t)+Ln[Riboflavin]₀ Equation 1.

where:
Ln=Natural logarithm
[Riboflavin]ₜ=Riboflavin concentration at time=t
[Riboflavin]₀=Riboflavin concentration at time=0 prior to light exposure
t=Light exposure time
k′=Pseudo-first order rate constant for Riboflavin

When pseudo-first order kinetics are present for riboflavin change, plotting Ln[Riboflavin]ₜ versus light exposure time thus yields a straight line, the slope of which is the desired pseudo-first order rate constant with units of inverse time. Dividing the natural logarithm of 2 by said constant (i.e., ln(2)/k′) yields the corresponding riboflavin half-life with units of time.

Riboflavin degradation rate constant values were calculated as described above using the linear regression calculation tools associated with Microsoft Excel and obtained correlation coefficient (R²) values were all 93% or greater indicating that the anticipated first order decay phenomena were observed.

### Package System Assessment

Five cylindrical, label-free bottles with tapered threaded openings made for the dairy drink market of the same shape and construction were chosen at random from a batch that was produced by a commercial bottle converter. Said bottles, derived from polyethylene terephthalate (PET) resin and containing titanium dioxide, possessed a bottom body diameter of 47 mm tapering to 34 mm at the bottle opening, a height of 130 mm, and a volume of about 194 mL. One of the bottles was designated as ‘Foil Wrapped Control’ and wrapped with light blocking aluminum foil up to the point where the exterior of the bottle transitioned to the subsequently applied bottle closure (the transfer bead), FIG. 10. The remaining bottles were designated as ‘1’ through ‘4’ and were not covered with aluminum foil, FIG. 9.

Each bottle, 30 in FIGS. 9 and 10, was filled with about 173 mL of cold (4° C.) 15 mg/L riboflavin solution (89 volume % filled), tightly capped with a light impermeable, rubber Suba Seal® septum (part number CG-3024-09; VWR, Radnor, Pa.) 31 in FIGS. 9 and 10, and finally affixed to an L-shaped support shelf, 32 and 33 in FIGS. 9 and 10, fashioned from a 30.48 cm long×10.16 cm wide×0.06 cm thick aluminum panel (Q-Lab, Westlake, Ohio) using a standard 5 mm wide, ultraviolet light resistant, nylon cable tie, 34 in FIGS. 9 and 10. The horizontal shelf, 33 in FIGS. 9 and 10, upon which each bottle rested had dimensions of 10.16 cm×7.62 cm and the securing cable tie, 34 in FIGS. 9 and 10, was positioned about halfway up the bottle length. Care was taken to minimize the light exposure prior to the desired light exposure to the bottle/support shelf assemblies as well as any time during the experiment where they were removed for measurement or assessment.

The bottle/support shelf assemblies, 28 and 37 in FIGS. 8-10, were placed into a dark 60° C. oven for 130 minutes to equilibrate the temperature of the packaged riboflavin solutions such that they would begin the Weather-Ometer light exposure at a temperature approximately the same as that of the Weather-Ometer exposure chamber interior temperature of 50° C. Maintaining consistent temperature profiles of the solutions with the pre-heating procedure was necessary as the light induced degradation kinetics of the solvated riboflavin are highly dependent upon temperature and thus by ensuring temperature consistency the experimental replication of the degradation kinetics is enabled. It is further noted that riboflavin solutions were confirmed to be stable with no degradation at the indicated temperatures in the complete absence of light and therefore the preheating procedures did not alter the riboflavin under the dark conditions utilized.

After the assemblies were removed from the oven, each closure was removed briefly to measure the temperature of the solution within each bottle using a handheld thermocouple (part number 61220-416, VWR) and the absorption spectrum of the riboflavin solution contained within each bottle under reduced laboratory lighting conditions.

To perform the RF measurements, the solution within each bottle was vigorously agitated by manually swirling the entire associated bottle/support shelf assembly for several seconds prior to the measurements. The Suba Seal® septum was removed, the RF concentration was measured, and the septum was immediately replaced. As each measurement was completed, each bottle/support shelf assembly was put inside the equilibrated Weather-Ometer (50° C.) and placed on the bottom ring of the rotatable Weather-Ometer sample carousel. Large, steel, paper binder clips secured each assembly in a manner that allowed each riboflavin solution filled bottle to fully face the centrally located Weather-Ometer xenon arc lamp for light exposure. The distance from the Weather-Ometer lamp center-of-mass to that of each bottle was about 43 cm. Light exposure commenced immediately after placement of the last assembly inside the Weather-Ometer chamber and closing of the chamber.

After 46 minutes of light exposure, the light exposure was paused. The chamber was opened and each bottle/support shelf assembly was rapidly and sequentially removed from the Weather-Ometer, the temperature and absorption spectrum of the associated riboflavin solution measured as previously described, and the assembly then placed back into the Weather-Ometer taking care to maintain the original orientation of each bottle relative to the Weather-Ometer light source. Each of the measurement cycles was completed within several minutes. Once the entire measurement process was complete for a given time interval of light exposure duration, the chamber was closed and the light exposure was resumed. This measurement process was repeated for accumulated light exposure durations of 100, 151, 204, and 245 minutes for the data set of replicate A. After several days, the entire light exposure experiment was replicated (replicate B) using the exact same packaging systems filled source of fresh riboflavin solution using the same procedures.

Packaged riboflavin solution absorbance data at 444.5 nm for each packaging system as a function of light exposure time for Replicate A is reported in Table 1. Said table also displays the corresponding solution temperature data averaged across all five bottles. Likewise, replicate B data is shown in Table 2. The data is shown in FIG. 11 further illustrating the consistency between the replications.

The data in Tables 1 and 2 were used to calculate the pseudo-first order rate constant for riboflavin degradation for each replication for each package system as described earlier using Equation 1. The data used for this analysis is shown in FIG. 12. An example of the analysis to determine the rate constant data is demonstrated in FIG. 13 for Bottle 1 where it is seen that the riboflavin decay profiles have excellent agreement to the pseudo-first order decay kinetics. Further it is seen that the data for the two replications are remarkably similar in their riboflavin decay profiles and associated pseudo-first order models demonstrating the repeatability of the method. The pseudo-first order riboflavin rate constants were similarly determined for each package system for both replications as presented in Table 3. These data demonstrate excellent repeatability of the method and determination of the rate constant data to characterize the photoprotective performance of each of the packaging systems. The data for each package system are averaged across Replication A and B as presented in Table 3 along with the corresponding calculated riboflavin half-lives.

TABLE 1

| | | Replication A: | | | | |
|---|---|---|---|---|---|---|
| | | Average Riboflavin Absorbance (444.5 nm), | | | | |
| | Average | Absorbance Units | | | | |
| Light | Riboflavin | | | | | Foil |
| Exposure | Solution | | | | | Wrapped |
| Time, | Temper- | Bottle | Bottle | Bottle | Bottle | Control |
| minutes | ature, ° C. | 1 | 2 | 3 | 4 | Bottle |
| 0 | 52.2 ± 0.9 | 0.252 | 0.253 | 0.252 | 0.253 | 0.250 |
| 46 | 48.4 ± 0.4 | 0.231 | 0.229 | 0.241 | 0.235 | 0.257 |
| 100 | 48.3 ± 0.3 | 0.199 | 0.189 | 0.218 | 0.203 | 0.255 |
| 151 | 48.7 ± 0.5 | 0.174 | 0.156 | 0.195 | 0.177 | 0.254 |
| 204 | 48.7 ± 0.5 | 0.142 | 0.122 | 0.175 | 0.145 | 0.253 |
| 245 | 48.8 ± 0.2 | 0.118 | 0.100 | 0.155 | 0.125 | 0.253 |

TABLE 2

| | | Replication B: | | | | |
|---|---|---|---|---|---|---|
| | | Average Riboflavin Absorbance (444.5 nm), | | | | |
| | Average | Absorbance Units | | | | |
| Light | Riboflavin | | | | | Foil |
| Exposure | Solution | | | | | Wrapped |
| Time, | Temper- | Bottle | Bottle | Bottle | Bottle | Control |
| minutes | ature, ° C. | 1 | 2 | 3 | 4 | Bottle |
| 0 | 52.0 ± 2.8 | 0.251 | 0.251 | 0.251 | 0.251 | 0.252 |
| 46 | 49.1 ± 0.3 | 0.227 | 0.223 | 0.237 | 0.231 | 0.247 |
| 100 | 49.4 ± 0.4 | 0.197 | 0.189 | 0.216 | 0.199 | 0.252 |
| 151 | 49.5 ± 0.3 | 0.168 | 0.153 | 0.195 | 0.176 | 0.261 |
| 204 | 49.6 ± 0.4 | 0.137 | 0.118 | 0.170 | 0.143 | 0.254 |
| 245 | 49.7 ± 0.2 | 0.126 | 0.107 | 0.154 | 0.127 | 0.260 |

TABLE 3

| Package | Pseudo-First Order Rate Constant, k¹ (min⁻¹) | | | Average Half- |
|---|---|---|---|---|
| System | Replication A | Replication B | Average | Life (min) |
| Bottle 1 | 0.0031 | 0.0029 | 0.0030 | 231 |
| Bottle 2 | 0.0039 | 0.0036 | 0.0037 | 185 |
| Bottle 3 | 0.0020 | 0.0020 | 0.0020 | 345 |
| Bottle 4 | 0.0029 | 0.0028 | 0.0029 | 240 |

Examination of the riboflavin solution temperature data provided in Table 1 reveals that during the initial light exposure experiment the five packaged riboflavin solutions entered the Weather-Ometer with an average temperature of 52.2±0.9° C. Said solutions then cooled within 46 minutes to an average temperature of 48.4±0.4° C. after which their average temperature remained largely invariant with a continued sub-one degree Celsius standard deviation for the remainder of said experiment. That the equilibrium temperature of the packaged riboflavin solutions was slightly below the 50° C. set point temperature of the Weather-Ometer light exposure chamber can be explained by postulating minor differences in the calibrations of the Weather-Ometer and the several handheld thermocouples used in this study.

Examination of the corresponding data provided in Table 2 reveals that during the repeat light exposure experiment the five packaged riboflavin solutions entered the Weather-Ometer with an average temperature of 52.0±2.8° C. Said solutions then cooled within 46 minutes to 49.1±0.3° C. after which their average temperature also remained largely invariant again with a continued sub-one degree Celsius standard deviation for the remainder of said experiment. Further optimization of the temperature set points could be performed to further reduce the temperature ranges, however, the procedures of the disclosed method demonstrate repeatable control of the temperature profiles across the replications.

Importantly, the above described data demonstrate that temperature differences among the five packaged riboflavin solutions during the entirety of both Replication A and Replication B light exposure experiments was exceptionally low, especially past the 46-minute light exposure point, thus providing a high degree of confidence that factors other than temperature contribute to the observed light blocking performance differences discussed below. Said data also demonstrate the high reproducibility of the light exposure experimental technique from a packaged riboflavin solution temperature standpoint. Thus, the control of a uniform and defined temperature within a replication and the temperature between replications is demonstrated.

Inspection of the absorbance data provided in Tables 1 and 2 for the foil wrapped control bottles and of the graphical representation of said data in FIGS. 11 and 12 reveal essentially unchanging, robust, and repeatable RF concentration profiles with exposure time for both the Replication A and Replication B light exposure experiments. The aluminum foil wrap around the control bottles and the use of light blocking septa as bottle closures in this package system essentially eliminated virtually any light exposure to the riboflavin solution within said bottles. This observation demonstrates that in the absence of light an aqueous solution of riboflavin will not undergo degradation at the temperatures associated with these light exposure experiments.

Reviewing the absorbance data provided in Table 1 for Bottles 1-4, it is obvious that the solvated riboflavin solutions contained within said bottles each experienced a measure of riboflavin degradation during the light exposure experiment. Referring to FIG. 12, it can be clearly observed that the rates of said degradation were not uniform across the bottle sample set. More specifically, the rate of solvated riboflavin degradation was observed to be slowest for Bottle 3, fastest for Bottle 2, while those of Bottles 1 and 4 were comparable to one another and between those of the first two mentioned bottles. Importantly, an examination of the absorbance data for Bottles 1-4 provided in Table 2 and of the graphical depiction of said data in FIG. 11, FIG. 12 and FIG. 13 show that the riboflavin degradation rate behavior observed for each of said bottles during the Replication A experiment was very closely duplicated during the Replication B experiment illustrating the repeatability of this method.

The above described riboflavin degradation rate behavior is quantified in Table 3 which also includes the average half-lives for riboflavin degradation which were calculated to be 345 minutes, 185 minutes, 231 minutes, and 240 minutes for Bottles 3, 2, 1, and 4, respectively. The observation of significantly different riboflavin degradation rates and half-lives across the evaluated bottle sample set was unanticipated as revealed by the novel data provided by this evaluation of the package system photoprotective performance. As the bottles were from a common product obtained from retail at the same time where all the evaluated bottles were apparently identical, presumably produced by the same bottle producer using the same production equipment and components, however, the data reveal that the bottles did not perform the same for photoprotective performance. In fact, the average riboflavin half-life data obtained from the method demonstrate a dramatic 46% discrepancy between Bottles 2 and Bottles 3 ((347 minutes−187 minutes)/347 minutes).

This example thus demonstrates that the light exposure technique discussed herein can be used to readily assess in accelerated fashion the photoprotective performance values of complete package systems. Because this technique integrates into one measurement the photoprotective performance behavior of all components of a packaging system (package body, closure, and possibly a label), it provides a more complete assessment of package photoprotective performance capability as compared to existing accelerated light block measurement methodology that can only evaluate package components on an individual, and thus separate, basis and does not allow for the performance of a complete and functional package system. Further this example demonstrates the sensitivity of this approach to discern photoprotective performance discrepancies for otherwise perceived identical packages thus providing a unique ability to discriminate performance of packages. This capability would be useful in manufacturing quality control as well as in risk assessment when designing products that require photoprotective packaging to maintain efficacy, potency, or other essential product attributes.

### Example 2

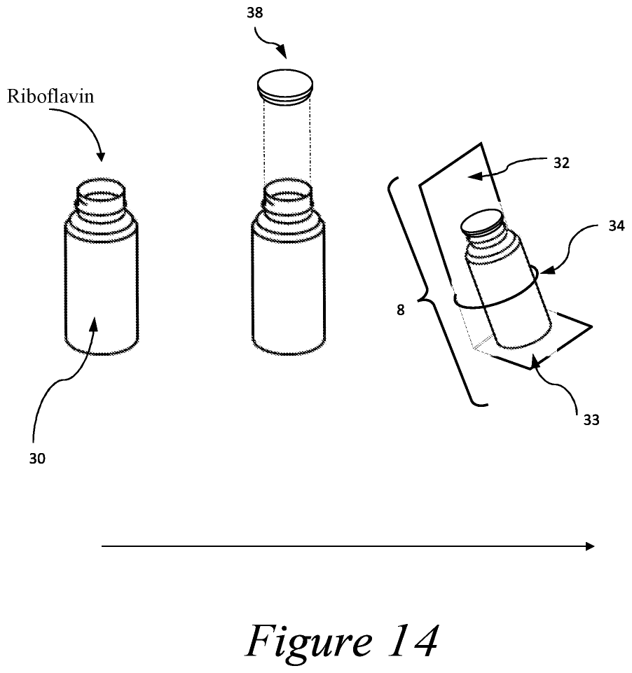

Example 2 may be best understood with reference to FIGS. 8 and 10, (previously described in Example 1) and FIGS. 14-16:

FIG. 14: Assembly of a Packaging System with Cap

According to an aspect of the invention, a package 30 is filled, sealed with a cap 38 and can be placed onto a holder consisting of a L-bracket with backing 32 and bottom shelf 33, with the filled package system equilibrated to a desired control temperature. The package is secured to the backing 32 by means of zip-tie 34. The filled, sealed with cap and secured package is referred to as a package assembly 8.

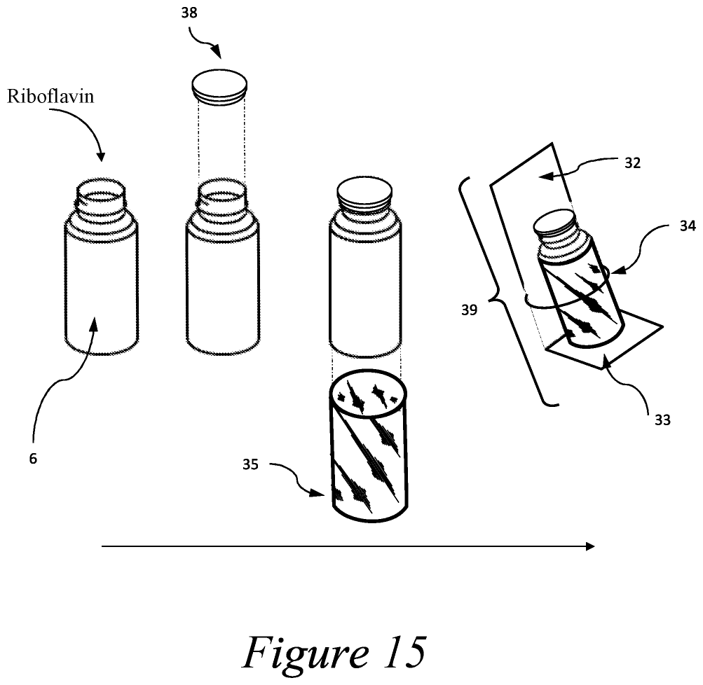

FIG. 15: Assembly of a Packaging System with Cap and Foil Sleeve

According to an aspect of the invention, a package 6 is filled, sealed with a cap 38 and further covered in foil 35 up to the point where the exterior of the bottle transitioned to the subsequently applied bottle closure (the transfer bead), and can be placed onto a holder consisting of a L-bracket with backing 32 and bottom shelf 33, with the filled package system equilibrated to a desired control temperature. The package is secured to the backing 32 by means of zip-tie 34. The filled, sealed with cap and secured package is referred to as a package assembly 39.

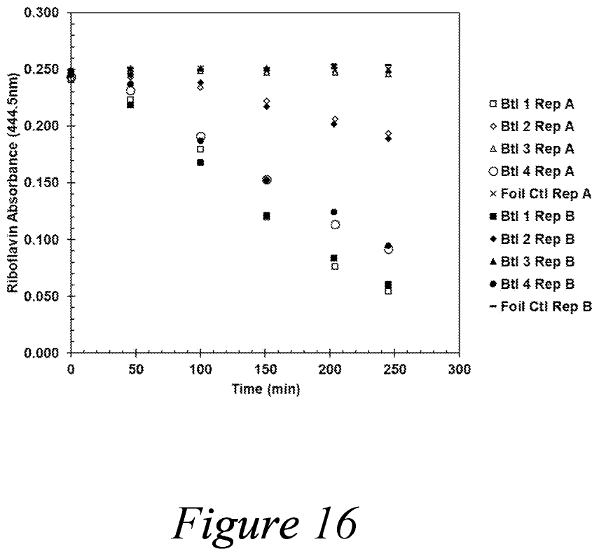

### FIG. 16: Natural Log of Riboflavin Absorbance Versus Light Exposure Time

FIG. 16 shows the decline in the natural logarithm (ln) of riboflavin absorbance for riboflavin solutions contained within package systems Bottle 1 (Btl 1, square symbols), Bottle 2 (Btl 2, diamond symbols), Bottle 3 (Btl 3, triangle symbols), and Bottle 4 (Btl 4, circle symbols) as a function of light exposure time. Open symbols are used for Replication A and closed symbols are used for Replication B. Also shown are data for the foil control package system (x, −) for Replication A and B, respectively.

The exact same five bottles and associated labeling scheme used in Example 1 were used in this example. Importantly, it should be recalled from Example 1 that the light blocking capability of the body of Bottle 1 is essentially identical to that of Bottle 4 as illustrated in the reported assessment data. Each of the five bottles was prepared for subsequent light exposure as indicated in Table 4 by creating a package system with bottle closures.

TABLE 4

| | Bottle Labeling | | |
|---|---|---|---|
| | (same bottles | Aluminum | |
| Package | as used in | Foil Bottle | Bottle |
| System Code | Example 1) | Wrap | Closure |
| Package | Bottle 1 | No | Non-light blocking |
| System 1 | | | polyethylene cap |
| Package | Bottle 2 | Yes | Non-light blocking |
| System 2 | | | polyethylene cap |
| Package | Bottle 3 | Yes | Light blocking |
| System 3 | | | polyethylene cap |
| Package | Bottle 4 | No | Light blocking |
| System 4 | | | polyethylene cap |
| Foil Control | Foil Wrapped | Yes | Light blocking rubber |
| | Control Bottle | | septum |
| | | | (same as used in |
| | | | Example 1) |

The aluminum foil wrap associated with Bottles 2 and 3 was applied in the same manner as for the foil wrapped control bottle, i.e. said wrap enclosed the entire body of said bottles up to the point where the exterior of each bottle transitioned to the subsequently applied bottle closure, FIG. 15. The two identically made, non-light blocking closures, one each for Bottles 1 and 2, were chosen at random from a batch that was produced by a commercial cap manufacturer. Said closures, derived from polyethylene and containing a blue colored pigment, were of the standard, 38 mm diameter, press-on type that is commonly used to seal polyethylene-based milk bottles and contained do light protecting ingredients. The two identically made, light blocking closures with light blocking ingredients, one each for Bottles 3 and 4, were also chosen at random from a batch that was produced by a different commercial cap manufacturer, all closures represented as 38 in FIGS. 14 and 15. These latter closures, also derived from polyethylene but containing a blue colored pigment, titanium dioxide, and carbon black, were also of the standard, 38 mm diameter, press-on type. Each of the four closures had its security tab and ring removed prior to commencing the light exposure experiment.

Each bottle was filled with about 173 mL of cold (4° C.) 15 mg/L riboflavin solution (89 volume % filled). Bottles 1 through 4 were then immediately and tightly capped with their respective closures rendering them filled packaging systems, see Table 4 and FIGS. 14 and 15, while the foil wrapped control bottle was sealed using the same light impermeable rubber septum as was used in Example 1 rendering it the light protected control filled packaging system, FIG. 10.

The support shelf mounting, pre-heating, light exposure FIG. 8, and temperature/riboflavin absorption measurement processes described in Example 1 were then promptly carried out on the filled packaging systems. One day later, the entire light exposure experiment was replicated using the exact same bottles, closures, and source of fresh riboflavin solution.

Packaged riboflavin solution absorbance data at 444.5 nm for each package system as a function of light exposure time for Replication A exposure experiment are shown in Table 5. Said table also displays the corresponding solution temperature data but averaged across all five bottles. Table 6 shows the same data as Table 5 but derived from Replication B light exposure experiment. The absorbance versus time data in Tables 5 and 6 were used to calculate an average (across both light exposure experiments) pseudo-first order rate constant for riboflavin degradation for Packaging Systems 1-4; said constants are shown in Table 7 along with corresponding calculated riboflavin half-lives. Graphical depiction of the absorbance versus time data contained within Tables 5 and 6 is provided in FIG. 16.

TABLE 5

| | | Replication A Light Exposure Experiment: | | | | |
|---|---|---|---|---|---|---|
| | | Average Riboflavin Absorbance (444.5 nm), | | | | |
| | Average | Absorbance Units | | | | |
| Light | Riboflavin | | | | | Foil |
| Exposure | Solution | Packaging | Packaging | Packaging | Packaging | Wrapped |
| Time, | Temperature, | System | System | System | System | Control |
| minutes | ° C. | 1 | 2 | 3 | 4 | Bottle |
| 0 | 47.9 ± 1.0 | 0.241 | 0.246 | 0.247 | 0.244 | 0.249 |
| 46 | 49.9 ± 0.5 | 0.223 | 0.243 | 0.249 | 0.232 | 0.250 |
| 100 | 50.1 ± 0.4 | 0.180 | 0.234 | 0.249 | 0.191 | 0.251 |
| 151 | 50.1 ± 0.6 | 0.120 | 0.222 | 0.248 | 0.153 | 0.248 |
| 204 | 49.9 ± 0.8 | 0.076 | 0.207 | 0.247 | 0.113 | 0.250 |
| 245 | 50.1 ± 0.4 | 0.055 | 0.194 | 0.246 | 0.092 | 0.250 |

TABLE 6

| | | Replication B Light Exposure Experiment: | | | | |
|---|---|---|---|---|---|---|
| | | Average Riboflavin Absorbance (444.5 nm), | | | | |
| | Average | Absorbance Units | | | | |
| Light | Riboflavin | | | | | Foil |
| Exposure | Solution | Packaging | Packaging | Packaging | Packaging | Wrapped |
| Time, | Temperature, | System | System | System | System | Control |
| minutes | ° C. | 1 | 2 | 3 | 4 | Bottle |
| 0 | 51.1 ± 1.9 | 0.246 | 0.245 | 0.246 | 0.249 | 0.248 |
| 46 | 48.8 ± 0.5 | 0.219 | 0.245 | 0.251 | 0.237 | 0.251 |
| 100 | 49.0 ± 0.7 | 0.168 | 0.239 | 0.251 | 0.187 | 0.250 |
| 151 | 49.1 ± 0.8 | 0.122 | 0.217 | 0.251 | 0.152 | 0.251 |
| 204 | 49.0 ± 0.5 | 0.084 | 0.202 | 0.253 | 0.124 | 0.254 |
| 245 | 48.8 ± 0.5 | 0.061 | 0.189 | 0.250 | 0.094 | 0.254 |

TABLE 7

| | Packaging | Packaging | Packaging | Packaging |
|---|---|---|---|---|
| | System 1 | System 2 | System 3 | System 4 |
| Average | 0.0061 ± | 0.0011 ± | 0.0000 ± | 0.0041 ± |
| Pseudo-First | 0.0003 | 0.0001 | 0.0001 | 0.0001 |
| Order Rate | | | | |
| Constant, min⁻¹ | | | | |
| Average | 114 | 630 | >>630 | 169 |
| Riboflavin Half- | | | | |
| Life, min | | | | |

Examination of the riboflavin solution temperature data provided in Table 5 reveal that during the initial light exposure experiment the five packaged riboflavin solutions entered the Weather-Ometer with an average temperature of 47.9±1.0° C. Said solutions then warmed within 46 minutes to an average temperature of 49.9±0.5° C. after which their average temperature remained largely invariant with a continued sub-one degree Celsius standard deviation for the remainder of said experiment. Examination of the corresponding data provided in Table 6 reveals that during the repeat light exposure experiment the five packaged riboflavin solutions entered the Weather-Ometer with an average temperature of 51.1±1.9° C. Said solutions then cooled within 46 minutes to 48.8±0.5° C. after which their average temperature also remained largely invariant again with a continued sub-1° C. standard deviation for the remainder of said experiment. As with Example 1, the above described data demonstrate that temperature differences among the five packaged riboflavin solutions during the entirety of both light exposure experiments was exceptionally low, especially past the 46 minute light exposure point, thus again providing a high degree of confidence that factors other than temperature contribute to the observed light blocking performance differences discussed below. Said data also further demonstrate the high reproducibility of the light exposure experimental technique from a packaged riboflavin solution temperature standpoint.

Inspection of the absorbance data provided in Tables 5 and 6 for the foil wrapped control bottles and of the graphical representation of said data in FIG. 16 reveals essentially unchanging riboflavin concentration as a function of exposure time for both Replicate A and B light exposure experiments. This observation provides further confirmation that in the absence of light an aqueous solution of riboflavin will not undergo degradation at the temperatures associated with these light exposure experiments.

A review of the of the absorbance data provided in Table 5 and of the graphical depiction of said data in FIG. 16 for Packaging Systems 1-4 demonstrates that the solvated riboflavin solutions contained within said bottles each experienced a different degree of riboflavin degradation during the initial light exposure experiment. More specifically, essentially no riboflavin degradation was observed for Packaging System 3 (aluminum foil bottle wrap plus light blocking closure) while a noticeable rate of riboflavin degradation was observed for each of the remaining three bottles with that for Packaging System 2 (aluminum foil bottle wrap plus non-light blocking closure) being the slowest, that for Packaging System 1 (no aluminum foil bottle wrap plus non-light blocking closure) being the fastest, and that for Packaging System 4 (no aluminum foil bottle wrap plus light blocking closure) positioned intermediate between those of the latter two bottles.

A further review of the corresponding absorbance data provided in Table 6 and of the graphical depiction of said data in FIG. 16 shows that the riboflavin degradation rate behavior observed for each of said packaging system during the initial light exposure experiment was very closely duplicated during the repeat experiment. The above described riboflavin degradation rate behavior is quantified in Table 7: the average half-lives for riboflavin degradation were calculated to be about 630 minutes, 114 minutes, and 169 minutes for Packaging Systems 2, 1, and 4, respectively, while the corresponding half-life for Packaging System 3 was incalculable due to the lack of statistically significant riboflavin degradation.

The different solvated riboflavin degradation half-lives calculated for Packaging Systems 1 and 4 (no aluminum foil wrap for either bottle both of which possess essentially identical light blocking capability, each with a differently performing light blocking closure) and also for Packaging Systems 2 and 3 (each possessing an aluminum foil wrap, each also with a differently performing light blocking closure) thus demonstrate the ability of the light exposure technique described herein to assess in accelerated fashion the comparative light blocking capabilities of bottle closures independent of bottle body while said closures are part of a complete package system.

### Example 3

Example 3 may be best understood with reference to FIG. 8 (previously described in Example 1) and 17-19:

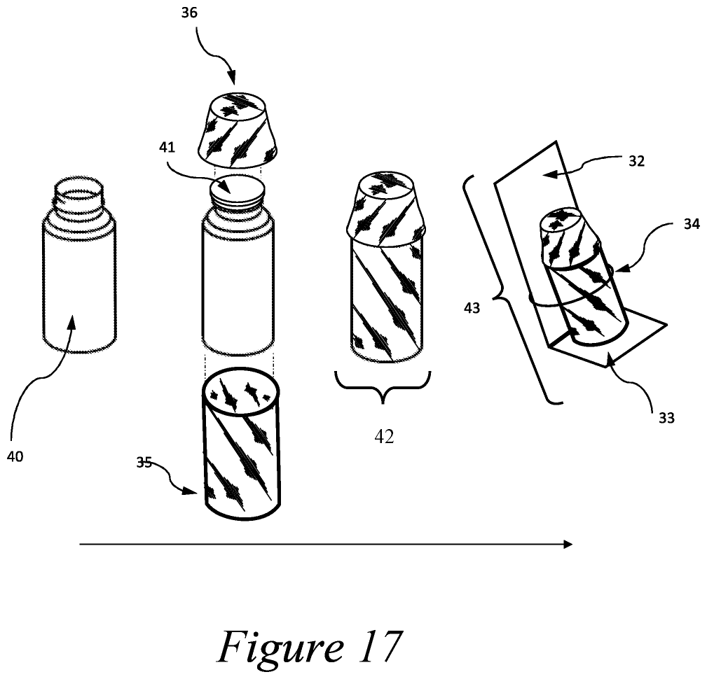

FIG. 17: Assembly of a Foil Packaging System with Cap

According to an aspect of the invention, a commercial package 40 is filled, sealed with its commercial cap 41 and further covered in foil 35 up to the point where the exterior of the bottle transitioned to the subsequently applied bottle closure (the transfer bead) and further capped with foil top 36. This foil package system is referred to as a package assembly 42. Package assembly 42 can be placed onto a holder consisting of a L-bracket with backing 32 and bottom shelf 33, with the filled package system equilibrated to a desired control temperature. The package is secured to the backing 32 by means of zip-tie 34. The filled, sealed with cap and secured package is referred to as a package assembly 43.

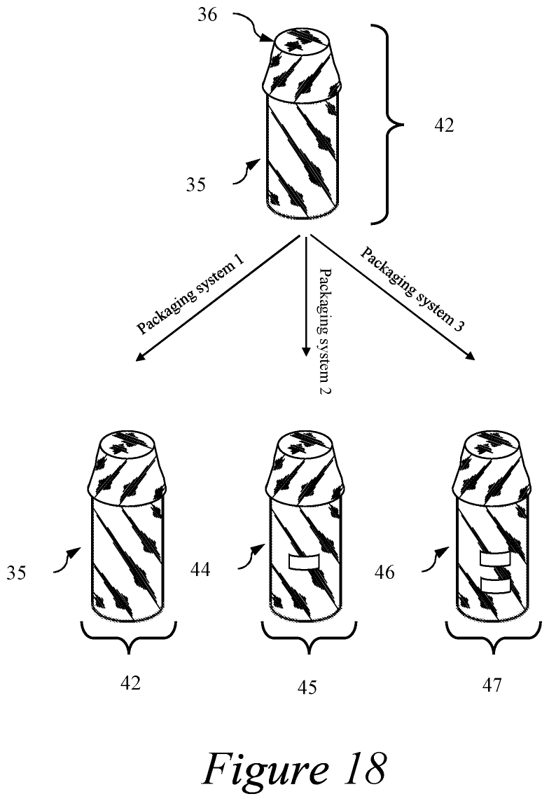

FIG. 18: Foil Covered Packaging Systems with Controlled Defects

According to an aspect of the invention, foil packaging system with cap, 42 FIG. 17, is modified in 1 of three ways to test for defects: 1) no modifications, 42, referred to as packaging system 1; 2) a rectangular cut is made in the foil sleeve, 44, the resulting packaging system, 45, referred to as packaging system 2; and 3) two rectangular cuts are made into the foil sleeve, 46, the resulting packaging system, 47, referred to as packaging system 3.

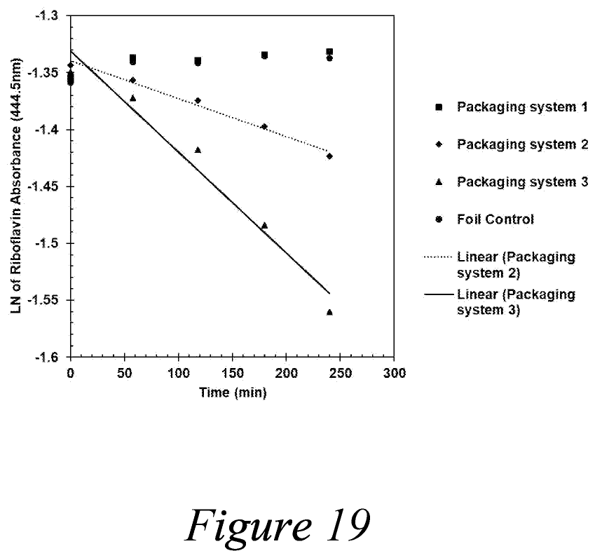

### FIG. 19 Natural Log of Riboflavin Concentration Versus Light Exposure Time

FIG. 19 shows the decline in the natural logarithm (ln) of riboflavin absorbance for riboflavin solutions contained within package systems 1 (square symbols), 2 (diamond symbols), 3 (triangle symbols), and Foil control (circle symbols) as a function of light exposure time. Also shown are data for the linear regression of the data for package system 2 (dashed line) and package system 3 (solid line).

Four identical, cylindrical, label-free bottles, 40 in FIG. 17, made for the dairy drink market were chosen at random from a batch that was produced by a commercial bottle converter. Each of said bottles was derived from high density polyethylene resin. The bottles had a bottom diameter of 54 mm (34 mm at the bottle closure end), a length of 127 mm, and a volume of about 187 mL. One of the bottles, designated as ‘Foil Wrapped Control’, was wrapped with light blocking aluminum foil, 35 in FIG. 17, up to the point where the exterior of the bottle transitioned to the subsequently applied bottle closure, 41 in FIG. 17, and foil cap, 36 in FIG. 17. The remaining bottles, designated as ‘1’ through ‘3’, were similarly wrapped with aluminum foil rendering them a packaging system, 42 in FIG. 17. Then each packaging system was modified with a wrap ‘defect’ as indicated immediately below:
Package System 1, 42 in FIG. 18: no modification was performed (bottle is effectively a duplicate foil wrapped control).
Package System 2, 45 in FIG. 18: an approximately rectangular section of the aluminum foil wrap about 4.5 mm (vertical direction)×about 12 mm (horizontal direction) in size and located about 45 mm from the bottle bottom was removed thereby exposing an approximately 54 mm2 area of the bottle body structure underneath, 44 in FIG. 18.
Package System 3, 47 in FIG. 18: two approximately rectangular sections of the aluminum foil wrap, the first about 4.5 mm (vertical direction)×about 14 mm (horizontal direction) in size and located about 45 mm from the bottle bottom, the second about 5.2 mm (vertical direction)×about 14 mm (horizontal direction) in size and located about 60 mm from the bottle bottom, were removed thereby exposing in total an approximately 136 mm² sized area of the bottle body structure underneath, 46 in FIG. 18. Importantly, it should be noted that the light blocking performance of the body of this bottle was essentially identical to that of Packaging System 2.

Package Systems 1, 2, and 3 and the foil wrapped control bottle were all filled with about 167 mL of cold (4° C.) 15 mg/L riboflavin solution (89 volume % filled). All four bottle package systems were then immediately and tightly capped with their associated commercial closures which were then covered with aluminum foil hats to prevent light infiltration from the closure top and sides. With slight modification to time durations, the steps of support shelf mounting, pre-heating (100 minutes), light exposure, FIG. 8, and temperature/riboflavin absorption measurement performed at 0, 58, 118, 180, and 240 minutes of light exposure processes described in Example 1 were then promptly carried out on the filled package systems. Note that the aluminum foil ‘defects’ associated with Package Systems 2 and 3 were directed straight towards the light source when said bottles were under light exposure in the Weather-Ometer.

Packaged riboflavin solution absorbance data at 444.5 nm for each package system as a function of light exposure time for the light exposure experiment are shown in Table 8. Said table also displays the corresponding solution temperature data but averaged across all four package systems. The absorbance versus time data in Table 8 were used to calculate pseudo-first order rate constants for riboflavin degradation for Package Systems 1-3; said constants are shown in Table 9 along with corresponding calculated riboflavin half-lives. Graphical depiction of the absorbance versus time data contained within Table 8 is provided in FIG. 19.

TABLE 8

| | Average | Riboflavin Absorbance (444.5 nm), | | | |
|---|---|---|---|---|---|
| Light | Riboflavin | Absorbance Units | | | |
| Exposure | Solution | Package | Package | Package | Foil |
| Time, | Temperature, | System | System | System | Wrapped |
| minutes | ° C. | 1 | 2 | 3 | Control |
| 0 | 50.7 ± 1.0 | 0.258 | 0.261 | 0.259 | 0.257 |
| 58 | 49.3 ± 0.3 | 0.263 | 0.257 | 0.254 | 0.262 |
| 118 | 49.2 ± 0.4 | 0.262 | 0.253 | 0.242 | 0.261 |
| 180 | 49.4 ± 0.4 | 0.263 | 0.247 | 0.227 | 0.263 |
| 240 | 49.5 ± 0.2 | 0.264 | 0.241 | 0.210 | 0.263 |

TABLE 9

| | Package | Package | Package |
|---|---|---|---|
| | System 1 | System 2 | System 3 |
| Average Pseudo-First | 0.00000 | 0.00033 | 0.00089 |
| Order Rate Constant, | | | |
| min⁻¹ | | | |
| Average Riboflavin | >>2100 | 2100 | 779 |
| Half-Life, min | | | |

Examination of the riboflavin solution temperature data provided in Table 8 reveals that during the light exposure experiment the four package systems containing riboflavin solutions entered the Weather-Ometer with an average temperature of 50.7±1.0° C. Said solutions then cooled within 58 minutes to an average temperature of 49.3±0.3° C. after which their average temperature remained largely invariant with a continued sub-one degree Celsius standard deviation for the remainder of said experiment. As with Examples 1 and 2, the above described data demonstrate that temperature differences among the four packaged riboflavin solutions during the entirety of the light exposure experiment was exceptionally low, especially past the 58-minute light exposure point, thus again providing a high degree of confidence that factors other than temperature contribute to the observed light blocking performance differences discussed below.

Inspection of the absorbance data provided in Table 8 for the foil wrapped control package system and of the graphical representation of said data in FIG. 19 reveals essentially unchanging riboflavin concentration as a function of exposure time. This observation provides continued confirmation that in the absence of light an aqueous solution of riboflavin will not undergo degradation at the temperatures associated with these light exposure experiments.

A review of the of the absorbance data provided in Table 8 and of the graphical depiction of said data in FIG. 19 for Package Systems 1-3 demonstrates that the solvated riboflavin solutions contained within said package systems each experienced a different degree of riboflavin degradation during the light exposure experiment. More specifically, essentially no riboflavin degradation was observed for Package System 1 (intact aluminum foil bottle wrap) while a noticeable rate of riboflavin degradation was observed for each of the remaining two package systems with that for Package System 2 (aluminum foil bottle wrap with about 54 mm² of said wrap removed) being slower versus that for Package System 3 (aluminum foil wrap with about 136 mm² of said wrapped removed) being the fastest. The above described riboflavin degradation rate behavior is quantified in Table 9: the half-lives for riboflavin degradation were calculated to be about 2100 minutes and 779 minutes for Package Systems 2 and 3, respectively, while the corresponding half-life for Package System 1 was incalculable due to the lack of statistically significant riboflavin degradation. Given that Package Systems 2 and 3 inherently possess comparable light blocking performances, the calculated half-life results for said package systems can be seen (as expected) to correlate in inverse fashion to the size of their corresponding aluminum foil wrap defects. Thus, Package System 2 possesses the longest half-life (2100 minutes) and the smallest defect area size (54 mm²) while Package System 3 possesses the shortest half-life (779 minutes) and the greatest defect area size (136 mm²).

The data presented in this experiment thus demonstrate the ability of the light exposure technique described herein to assess and quantify in accelerated fashion the comparative photoprotective performance capabilities of package systems including those with layers such a primary container like bottles further including bottle wraps or labels. In this example, the effects of defects in a layer of a package system are assessed while said layers are part of a complete package system.

### Example 4

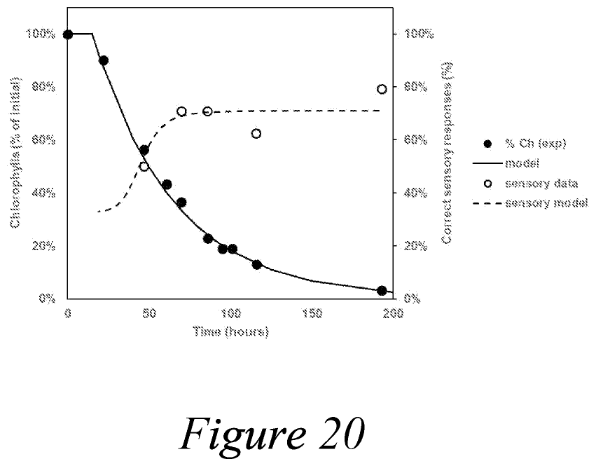

Example 4 may be best understood with reference to FIGS. 1-2 (previously described in the specification and FIG. 20:

### FIG. 20. Chlorophyll Content and Correct Sensory Response of Packaged Olive Oil as a Function of Light Exposure Time

FIG. 20 shows the chlorophyll content decline in light exposed packaged olive oil in filled circles on the primary axis with a solid line indicating the model for the first order reaction kinetics of this decline. On the secondary axis, the percentage of correct sensory responses for the evaluation of this olive oil is shown with the model for the growth in this behavior indicated with a dashed line.

In this example, the inventive device and method of this patent application are used to quantify the performance of an olive oil package system. The photoprotective performance of the package system was evaluated by exposing the package system containing olive oil to defined light exposure, tracking the change to a key photosensitive species found in olive oil, and also by monitoring changes to the sensory quality of the olive oil. This was done by providing defined light exposure to the package system using an accelerated retail isothermal exposure device and then evaluating the olive oil for its properties.

### Packaged Olive Oil

Twelve, rectangular prism shaped green glass bottles (23.5 cm×16.2 cm×11.4 cm, 500 mL in volume) with a screwed opening and metallic closure containing extra virgin olive oil product of a common olive oil brand and variety (California Olive Ranch, Everyday Fresh California Extra Virgin Olive Oil) were purchased at retail (Target, Wilmington Del.). All bottles were selected from the same harvest year. Six pairs of bottles were matched from the same batch, as determined by the lot numbers indicated on the labels, to ensure consistency of the olive oil product within the paired samples. The labels were removed from the exterior of all 12 bottles while they remained sealed, shown generally as 6 in FIG. 2.

For each of the 6 bottle pairs of olive oil product, one bottle was wrapped in foil and designated as the dark control sample, referred to as the “control” bottle while the other bottle was left uncovered and referred to as the “test” bottle. These two bottle types, control and test, were light exposed together in pairs for a defined duration of time under controlled light exposure conditions and evaluated for photo protective performance.

### Evaluation of Olive Oil Package System

Chlorophyll is a known photosensitizer in foods that generates reactive oxygen species through light-driven energy transitions.^{1,2} Besides generating reactive oxygen species, chlorophyll itself is decomposed by the same light-generated species and its concentration can be tracked by monitoring the light absorbance at 670 nm of a chlorophyll-containing species.

In this example, the filled package system under evaluation consisted of olive oil in green glass bottles. Chlorophyll content in the packaged olive oil was tracked as an indicator of change to the olive oil as a result of light exposure. Chlorophyll was measured with HunterLab UltraScan PRO visible spectrophotometer in transmission mode. The instrument was always standardized according to the manufacturer's protocol prior to measurement. For each measurement, approximately 15 mL of olive oil was poured from the package system into a rectangular quartz transmission cell with a 10 mm path length and then the cell was placed within the instrument. The transmission spectrum was recorded and the absorbance at 670 nm was calculated according to the following equation:

Absorbance₆₇₀=−log(% Transmittance₆₇₀) (Equation 1)

The chlorophyll content retained in the sample at a given light exposure time is presented as a fraction versus the chlorophyll content at the initial time. The percent chlorophyll retained was then calculated from the absorbance at 670 nm before any exposure time (t₀) according to the equation:

$$\% chlorophyll = \frac{{Absorbance}_{670}}{{Absorbance}_{670} at {t}_{0}} \qquad ( Equation 2 )$$

In addition to chlorophyll content, sensory evaluations were also performed on the light exposed olive oil samples as further described below.

### Light Exposure Chamber and Package Positioning

The light exposure chamber depicted in FIGS. 1 and 2 was assembled to provide an isothermal light exposure system that would spectrally mimic retail light but at much enhanced light intensity allowing for an accelerated light dose to be delivered to the package systems. The light provided was visible light with a spectrum relevant to retail sources yet emitted at a light intensity greater than a magnitude higher than that of a typical retail lighting environment.

The light exposure chamber device used for this study is shown in FIGS. 1 and 2 and is comprised of the following components: one VBENLEM 98L capacity countertop display refrigerator (Amazon.com), 1, with a temperature controller, 3, capable of regulating temperature from 0° C. to 25° C. The rectangular refrigerated light exposure chamber has built in light sources comprising four vertical light strips oriented along each vertical corner as well as one overhead light in the ceiling of the chamber. The chamber was fitted with two shelves (denoted A and B), each with approximately 30 cm of clearance above it and upon which a white, 10″ Fotoconic electric motorized rotating turntable (Amazon.com), 7, was placed. The turntables were set to rotate at a cadence of 1 revolution per 40 seconds (0.025 Hz). The side walls of the exterior of the light exposure chamber comprised of transparent glass walls which were wrapped in reflective sheeting, 4, to isolate the light exposure environment from exterior lighting and to increase the interior light flux within the chamber. Five sets of 5-meter long dimmable white LED MINGER light strips (Amazon.com), 5, each with its own power adapter and controller, were affixed to line the inner side walls, ceiling, and bottom side of the top shelf in a uniform array to provide substantial illumination toward the interior of the chamber.

Each of the five light strips can be set to each of 6 levels (including off as a level) in addition to the 2 levels of the corner lights being turned on or off. The flexibility in the use of these light settings offers a large range of discrete light intensity environments to meet the needs of an experiment. The chamber light intensity was fully characterized with these settings to allow for the desired light intensities to be set for the experimental objectives. A partial set of data from this characterization is given in Table 10 to illustrate the 5 of the possible light intensity environments for each shelf (A, B) fixing all light five strips to the light setting denoted in “light strip settings”. The settings within the chamber can be adjusted based on this known characterization to target desired light doses.

For the present example, this chamber characterization enabled the use of intermediate settings to achieve the desired intensity for this study of 15,800 lux at the package system position as confirmed with sensor monitoring concurrent with the study.

TABLE 10
Characterization of AR Light Chamber Device. Settings and
Intensity with Corner lights on in the absence of packages

| | Average Light Intensity | |
|---|---|---|
| | at Position (lux) | |
| Light Strip Settings | Shelf A | Shelf B |
| 1 | 6,404 | 6,016 |
| 2 | 10,135 | 9,521 |
| 3 | 13,893 | 13,125 |
| 4 | 17,459 | 16,385 |
| 5 | 21,766 | 20,514 |

For light exposures, the package systems were arranged uniformly adjacent to their paired sample (e.g., test and control) about the circumference of the rotating turntables to provide a uniform light dose exposure to packages from the lights arranged about the chamber. In this study, the packages were distributed with 6 packages on each of the two shelves in the placements described below.

### Light Exposure of Package Systems

Six bottles were light-exposed in their native packaging (i.e. no alteration beyond removing the labels), referred to as “test” bottles. Half of the bottles were placed on the top shelf (Shelf A) and half on the bottom shelf (Shelf B) of a light exposure chamber as shown in FIG. 2, with an even mix of control and test bottles on each. The bottles were labeled from 1 to 12, with odd numbers for control bottles and even numbers for test bottles. Bottles 1, 2, 3, 4, 9, and 10 were placed on the top shelf at the beginning of the experiment, with the remaining bottles (Bottles 5, 6, 7, 8, 11, 12) on the lower shelf. Every 24 to 48 hours of exposure the bottles on the top shelf were swapped with bottles on the bottom shelf to ensure equivalent light dose over the course of the experiment.

The chlorophyll content was periodically measured in Bottles 1 and 2 (a control and test pair) to monitor chlorophyll degradation. All other bottles remained sealed throughout the duration of exposure. The chlorophyll levels in Bottles 1 and 2 were assumed to be representative of the chlorophyll levels in corresponding control and test bottles with Bottle 1 representative of all control bottles and Bottle 2 representative of all test bottles, as previous experiments confirmed this to be a reasonable assumption.

With accumulated light exposure, the chlorophyll content in Bottle 2 was found to decline. When the chlorophyll level in Bottle 2 reached target levels of chlorophyll retention (chlorophyll level of 56%, 36%, 23%, 13%, and 3% of initial levels) due to light exposure, a pair of control and test bottles were removed from the light exposure device and reserved for sensory evaluation with care to limit additional light exposure.

Bottles 1 and 2 remained in the exposure device throughout the experiment. During the light exposure, temperature within the light exposure device was measured to be maintained at 22±4° C. and light intensity was measured to be maintained at an average of 15,800 lux, as determined by periodic measurements.

Sample numbers, bottle types, exposure times, chlorophyll level at the time of removal from the light exposure chamber and accumulated light dose data are all reported in Table 11.

TABLE 11
Light exposure dose data

| | | Bottle 1 | Bottle 2 |
|---|---|---|---|
| | Accumulated | chlorophyll** | chlorophyll** |
| Exposure time | light dose* | (% of initial) | (% of initial) |
| (hours) | (klux-hours) | Control | Test |
| 0 | 0 | 100% | 100% |
| 22 | 348 | 100% | 90% |
| 47 | 743 | 102% | 56% |
| 61 | 964 | 99% | 43% |
| 70 | 1106 | 99% | 36% |
| 86 | 1359 | 99% | 23% |
| 95 | 1501 | 93% | 19% |
| 101 | 1596 | 99% | 19% |
| 116 | 1833 | 99% | 13% |
| 193 | 3049 | 99% | 3% |

*Accumulated light dose is calculated by multiplying the exposure time by the average measured light flux (15.8 klux).
**This equal to the bottle's olive oil absorbance at 670 nm at the time sampling divided by the absorbance at 670 nm before exposure began.

### Sensory Evaluations

Five sets of bottles, each consisting of one control and one test bottle, with identical light exposure times and accumulated light dose, were sent to a third-party vendor specializing in the sensory evaluation of olive oils (Applied Sensory, California) applying best practices and defined protocols for sensory evaluations using trained panelists. A difference test was conducted by tasting olive oil from each set of bottles. Each difference test consisted 8 to 15 trained panelists that conducted one to three triangles tests each, with a total of 24 triangle tests per difference test.

In each triangle test, a set of three approximately 12-13 mL samples of olive oil was poured from the test and control packages into blue glasses specified by the International Olive Oil Council according to method COI-T20_Doc 5 Rev_1 2007, so as to render the oil samples identical in terms of appearance. The glasses were covered with plastic lids, labeled with random 3-digit number codes, and placed on metal serving trays. Panelists were presented with the three blinded samples and instructed that, “Two oils are the same and one is different. Please evaluate all three samples from left to right then circle the code of the one sample that is DIFFERENT from the other two.”

As per standard triangle testing protocols, in some triangle sample sets, there were two control and one test sample, and in other triangle sample sets there were one control and two test samples. When the different sample was correctly identified the evaluation was marked as correct. The data is tabulated below (Table 12). The percent of correct responses was used to gauge whether the samples were distinguishable beyond random chance (33% chance of randomly guessing correctly in a triangle test) and the significance level (p-value) of correct responses was calculated for each pair of samples. When the significance level was greater than 5%, the null hypothesis, that there was no discernible difference between control and test samples, was accepted (i.e. >5% of the time the percent of correct responses could be obtained by chance alone). The p-values of correct responses in the difference tests were calculated according to the methodology described by Lawless & Heymann.³

### Results & Study Conclusions

The chlorophyll levels and light doses for Bottles 1 and 2 over the period of exposure are summarized in Table 11. The accumulated light dose was calculated by multiplying the exposure time by the average light flux measured throughout the experiment (15.8 klux). The chlorophyll in Bottle 1 remained unchanged (100%±2) throughout the exposure period; as anticipated, the foil wrap provided full light protection and prevented light damage to the olive oil product. Inspection of the data in Table 11 show that the chlorophyll in Bottle 2 decreased with light exposure time in a manner consistent with pseudo-first order reaction kinetics and consistent with previous studies of photosensitizers in food systems.⁴ The data can be further used to determine the pseudo-first order rate constant (k′) for the level of chlorophyll (Ch) as a function of light exposure time (t) in this package system under these light exposure conditions using the following equation where tᵢ is the induction time for the onset of the chlorophyll degradation to begin and Ch(0) is the initial level of chlorophyll.

Ch(t)=Ch(0)exp[−k′(t−tᵢ)] (Equation 3)

The Ch(t) data is presented as a percentage where the initial value of chlorophyll, Ch(0), is 100%. Applying this model, it is found that k′ is 0.02 hours⁻¹ and tᵢ is 15 hours. These data are graphically shown in FIG. 20 illustrating the onset of the first order decay of chlorophyll with first order kinetics after about 15 h of light exposure. For the complex system of olive oil, the delayed onset of the light induced degradation of chlorophyll may be due to the presence of antioxidants or other species that protect the chlorophyll from light degradation for a period of time. The decay is then found to proceed with the decay behavior indicative of pseudo first order reaction kinetics. The profile is well described by Equation 3 as shown by the solid line in FIG. 20 showing the model fit.

The chlorophyll contents in Bottles 1 and 2 were assumed to be representative of chlorophyll levels in other control and test bottles, respectively, under the same light dose conditions. For example, Bottle 4 was removed from the system after 47 hours, at which point chlorophyll levels in Bottle 2 were 56% of initial. It was then assumed that the chlorophyll in Bottle 4 was 56%. The bottle type (test, control), exposure time, chlorophyll levels, and accumulated light doses for each bottle are shown below in Table 12. Control and test bottles were removed from the system in pairs to provide sensory evaluation samples with identical light exposure times.

TABLE 12
Exposure data for package systems

| | | Light | Chlorophyll | Accumulated |
|---|---|---|---|---|
| | Bottle | Exposure | Retention* | light dose** |
| Sample ID | type | Time (hours) | (% of initial) | (klux-hours) |
| Bottle 1 | Control | 193 | 99 | 3049 |
| Bottle 2 | Test | 193 | 3 | 3049 |
| Bottle 3 | Control | 47 | 102 | 743 |
| Bottle 4 | Test | 47 | 56 | 743 |
| Bottle 5 | Control | 70 | 99 | 1106 |
| Bottle 6 | Test | 70 | 36 | 1106 |
| Bottle 7 | Control | 86 | 99 | 1359 |
| Bottle 8 | Test | 86 | 23 | 1359 |
| Bottle 9 | Control | 116 | 99 | 1833 |
| Bottle 10 | Test | 116 | 13 | 1833 |
| Bottle 11 | Control | 193 | 99 | 3049 |
| Bottle 12 | Test | 193 | 3 | 3049 |

*For test bottles, this is equal to Bottle 2's olive oil absorbance at 670 nm at the time of removal from the system divided by the absorbance at 670 nm before exposure began. For control bottles, it is the same method but using Bottle 1 as a reference.
**Accumulated light dose is calculated by multiplying the exposure time by the average measured light flux (15.8 klux).

The sensory evaluations were designed to test whether olive oil with the same exposure times but different light protection (control vs. test) were distinguishable by their sensory qualities of taste and aroma. The foil wrapped bottles were considered control bottles as no light degradation had occurred (chlorophyll=100±2%). The controls help confirm that degradation by other mechanisms, such as thermal oxidation, did not occur under study conditions, and that light exposure was the only impact to result in product change.

The results of the sensory evaluations are shown below in Table 13. When the olive oil was light exposed to an extent that only 3% of the initial chlorophyll remained, the difference between the test and control olive oil was correctly identified 79% of the time in the sensory testing. While the number of correct answers deviated with respect to chlorophyll levels, in all triangle tests the number of correct answers was greater than random chance (33% in a triangle test), indicating that for these panelists and these olive oils there was a distinct sensory difference between test and control packaged olive oils at all levels of light exposure. At 56% of initial chlorophyll the percent of correct responses dropped to 50%, indicating an inflection point in the response at this level and threshold of difference detectability. Furthermore, the significance level at 56% of initial chlorophyll in the test bottle was greater than 5%, beyond the threshold at which the null hypothesis can be rejected, leading to the conclusion that bottles 3 and 4 are not statistically discernible by sensory evaluation.

TABLE 13
Sensory evaluation data

| | | Chlorophyll | | | | |
|---|---|---|---|---|---|---|
| | | Level in | | # of | % | |
| Control | Test | test bottle | # of | triangle | Correct | Significance |
| Bottle | Bottle | (% initial) | panelists | tests | answers | Level |
| Bottle 3 | Bottle 4 | 56 | 8 | 24 | 50% | 6.8% |
| Bottle 5 | Bottle 6 | 36 | 10 | 24 | 71% | 0.1% |
| Bottle 7 | Bottle 8 | 23 | 8 | 24 | 71% | 0.1% |
| Bottle 9 | Bottle 10 | 13 | 15 | 24 | 63% | 0.3% |
| Bottle 11 | Bottle 12 | 3 | 15 | 24 | 79% | 0.1% |

The quantification of the light dose threshold for sensory detection of light exposure to a packaged olive oil provides a useful metric to quantify the photoprotective performance of the package. This metric can be used to design light-protective packaging that is tailored to the olive oil and lighting conditions the product is expected to endure. Thus, this data demonstrates the utility of the light exposure method and device for providing quantitative data to guide package design. Further the quantification of the impact of chlorophyll retention as a function of the light exposure time enabled by the devices and methods of the invention allow the data for these two metrics of quality to be compared for the package system under evaluation as shown in FIG. 20.

By using the methods and devices to generate data correlations as demonstrated in the data in FIG. 20, the level of chlorophyll could be used as a metric to predict sensory performance in future evaluations. This approach proved useful as the cost and expertise required to conduct sensory evaluations may not be an option for all package studies and thus having the ability to use the simple chlorophyll analysis can serve as a proxy to anticipate sensory performance for future studies.

This example demonstrates the utility of the light exposure device to be used to provide controlled light exposures simulating retail exposures to package systems. The light dose and temperature were well controlled and confirmed by monitoring of the conditions, using sensors 8 in FIG. 2, during the light exposure event as demonstrated by this example. Further, the example demonstrates how the light exposure device can be used in conjunction with the light exposure methods to evaluate the photoprotective performance of the package system.

This example illustrates how the methods and devices of the present invention can be useful to provide a defined light exposure to the packaged product, in this case an olive oil in a glass bottle with a metallic cap. This light exposure can be used to create defined light doses to a package system containing a product and allow for the characterization of the photoprotective performance of the package system and allow for the determination of metrics to quantify the photoprotective performance of the package system.

Here the photoprotective performance of the olive oil package system was quantified by determining the rate constant for the decline of chlorophyll after light exposure. The device of the invention was used to provide controlled and defined light doses to a packaged product. Specifically, here olive oil products were delivered defined doses of light to prepare a set of samples for further evaluation by sensory assessment. The sensory assessment data of this set of samples enabled the threshold of sensory quality decline associated with light dose to be determined and quantified. This threshold provides a quantitative metric for the package performance for light exposed olive oil applications. Such characterization of a package system for photoprotection performance, enabled by the devices and methods of this invention, allowed for a novel approach to determining the performances of a package system (green glass bottles with metallic caps) for photoprotection of package contents (olive oil) from accelerated retail light exposure under isothermal conditions by monitoring product qualities including chlorophyll retention and sensory quality stability.

### REFERENCES

(1) Min, D. B.; Boff, J. M. Chemistry and Reaction of Singlet Oxygen in Foods. Compr. Rev. Food Sci. Food Saf. 2002, 1 (2), 58-72. https://doi.org/10.1111/j.1541-4337.2002.tb00007.x.
(2) Krieger-Liszkay, A. Singlet Oxygen Production in Photosynthesis. J. Exp. Bot. 2004, 56 (411), 337-346.
(3) Lawless, H. T.; Heymann, H. Sensory Evaluation of Food: Principles and Practices; Springer Science & Business Media, 2013.
(4) Stancik, C. M.; Conner, D. A.; Jernakoff, P.; Niedenzu, P. M.; Duncan, S. E.; Bianchi, L. M.; Johnson, D. S. Accelerated Light Protection Performance Measurement Technology Validated for Dairy Milk Packaging Design. Packag. Technol. Sci. 2017, 30 (12), 771-780. https://doi.org/10.1002/pts.2326.

### Example 5

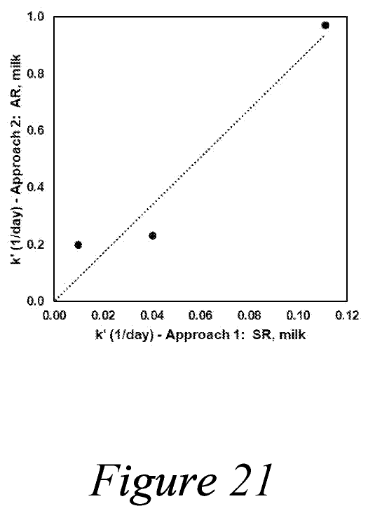

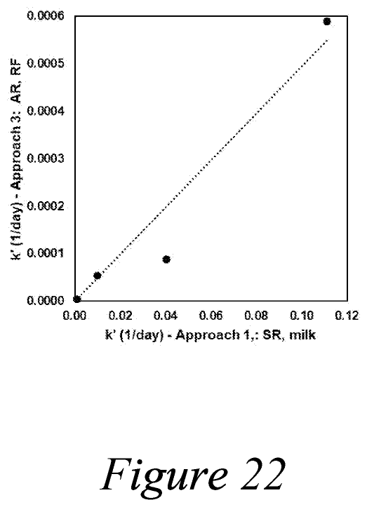

Example 5 may be best understood with reference to FIGS. 1-2 (previously described in the specification) and FIGS. 21-22:

### FIG. 21: Observed Pseudo First Order Rate Constants for Riboflavin Decline in Light Exposed Packaged Milk for a Set of Package Systems

FIG. 21 shows the data for the rate constants of riboflavin decline in milk determined for package systems exposed to light by two approaches: either Approach 1 of simulated retail (SR) (x-axis) or Approach 2 of accelerated retail (AR) (y-axis). The dashed line shows the linear regression for the correlation of the data by the two approaches.

### FIG. 22: Observed Pseudo First Order Rate Constants for Riboflavin Decline in Light Exposed Packaged Milk Compared to Light Exposed RF Solution for a Set of Package Systems

FIG. 22 shows the data for the rate constants of riboflavin decline in products in package systems exposed to light by accelerated retail (AR) for milk (x-axis) (Approach 1) and RF solution (y-axis) (Approach 3). The dashed line shows the linear regression for the correlation of the data by the two approaches.

In this example, a common set of package system conditions is evaluated and compared using three different approaches. In these approaches, two light exposure methods were used including simulated retail exposure with commercial refrigerated retail cases and the accelerated retail isothermal device, FIGS. 1 and 2, described in Example 4 with a storage temperature of 10 C. Within the approaches, two package contents systems were tracked for riboflavin content including fluid milk and an aqueous buffered riboflavin (RF) solution. This resulted in the three approaches as listed in Table 14. The goal of this example is to determine and correlate the photoprotection performances of the evaluated package systems across the approaches and demonstrate methods and devices of the present inventions.

TABLE 14

| Approach | Package Contents | Light Exposure Condition |
|---|---|---|
| Approach 1 | Milk | Simulated Retail (SR) |
| Approach 2 | Milk | Accelerated Retail (AR) |
| Approach 3 | RF solution | Accelerated Retail (AR) |

### Package System Conditions and Cleaning

Polyethylene terephthalate (PET) packages were produced by injection stretch blow molding with different levels of light protection pigments comprising surface treated titanium dioxide (TiO2, Ti-Pure™ R-104, The Chemours Company) and carbon black pigment (CB, CAS Reg. No. 1333-86-4). Five PET packaging conditions were created for the study as described in Table 15. Package dimensions for the resultant PET bottle conditions were uniform across the conditions and measured as follows.
Height to shoulder=12.0 cm
Height total=15.2 cm
Outer diameter of bottle cylinder=4.8 cm
Inner diameter of neck opening=2.4 cm
Total volume=224 mL to top of the neck

TABLE 15
PET Package Conditions

| | Light Protection | |
|---|---|---|
| Treatment | Performance | Light Protection Additives |
| N | None | None |
| M | Moderate | 3.7 wt % TiO2 |
| H | High | 6.9 wt % TiO2 |
| S | Superior | 6.8 w % TiO2; 23 ppm CB |
| F | Full | None, wrapped with foil |

Package closures for all package conditions were the same and were commercially obtained and were wrapped in foil rendering them fully light blocking. Additionally, Parafilm® (Sigma-Aldrich Inc, St. Louis, Mo.) was used to wrap over the bottle neck and the cap to improve sealing after filling for fluid milk contents in Approach 1 and 2. The bottles conditions with the package closures formed the package system conditions.

The day before package filling with contents, bottles and closures were rinsed with tap water and then soaked in sanitized solution for 10 sec. Then, all packaging bottles and caps were placed upside down for drying.

### Package Contents of Fluid Milk—Approach 1, Approach 2

For Approach 1, fresh raw milk was picked up from the Virginia Tech Dairy Science Complex-Kentland Farm (Blacksburg, Va.) and collected in three 5-gallon sanitized stainless-steel cans with caps. Raw milk was transported to the Virginia Tech Food Science and Technology dairy pilot plant within 15 min of collection and processed immediately. To limit the external light exposure, light was minimized in the pilot plant during milk processing and packaging, until packaged milk was placed into the controlled lighting conditions.

Before UHT (ultra-high-temperature) processing, milk was pre-warmed to 55° C. and separated into cream and skim milk using a pilot plant separator (model 1G, 6400 rpm, Bonanza Industries Inc., Calgary, Canada). Then milk was standardized to 2.0±0.1% fat, as verified by Babcock method (AOAC, 1995). Milk was homogenized in a 2-stage homogenizer (Type DX, Cherry Burrell Corp., Delavan, Wis.) and then heated at 131.1° C. for 2 s (UHT/HTST Lab-25 DH pasteurizer, MicroThermics, Raleigh, N.C.). Milk was rapidly chilled to room temperature by cool-heat-exchanger equipped on homogenizer, then the milk outflow was filled into the study package systems leaving a headspace of 20±5 mL for each bottle and the bottles were capped.

For Approach 2, a commercial source of standard UHT milk was obtained fresh from a commercial vendor ensuring minimal light exposures had been received prior to receipt.

Milk was clean-filled into PET packages under a positive laminar flow hood (Atmos-Tech Industries, Ocean, N.J.) to limit external contamination. Filled bottles were stored in a dark, refrigerated cooler (4 C±1 C) until transferred to the lighted-retail dairy case or accelerated retail light exposure device.

### Package Contents of Riboflavin Solutions—Approach 3

Riboflavin solutions were prepared as described in Example 1 and filled into the packages for evaluation.

### Simulated Retail (SR) Light Exposure Conditions—Approach 1

An open-front refrigerated retail case (Model O5DM, Hillphoenix, Chesterfield, Va.) equipped with LED lighting was used to simulate retail conditions in this study. The entire retail case was installed with the same type of LED light bulbs (Hillphoenix, Chesterfield, Va.) and the LED light appeared as neutral “daylight” white (3500 K, 10 watts). Temperature of the retail cases was maintained at 4° C.±1° C. throughout the experiment. Dark curtains were hung around the retail cases completely to minimize outside lighting.

Each packaging treatment (n=5) was tested in duplicate for each time treatment. Placement of PET bottles was randomized by JMP 13.0 Statistical Discovery Software (SAS Institute, Cary, N.C.) so that all treatments were distributed randomly in the retail case. Light intensity of individual milk bottles was monitored throughout the experiment to ensure each bottle received identical light exposure, as described by Wang et al. (2018). The average light intensity of tested milk bottles was 3823±263 lux for the SR exposures. PET packaging treatments were stored in this SR condition for five time intervals (0, 1, 2, 4, 13, 26 weeks) for each of three replications.

### Accelerated Retail (AR) Isothermal Light Exposure System—Approach 2

The light exposure system was used as described in Example 4. Twelve bottles of milk were placed into the accelerated retail isothermal exposure system uniformly on the rotating the carousal on each of two shelves. Duplicate samples of each package conditions (one of shelf A, one of shelf B) were exposed for intervals of light exposure. Light exposures were performed to produce samples with 1, 2, 4, 8, and 16 days of light exposure. After the desired light exposure dose had been received by each package condition, the milk contents of the package were swirled to ensure the contents were uniform and then transferred into storage centrifuge tubes (30 mL per a tube) and frozen until analysis. Samples were subsequently analyzed for riboflavin as described below. The light intensity and temperature were monitored in the device during the duration of the light exposure period using a Bluetooth Low Energy (BLE) capable, portable light sensor (HOBO Pendant MX Temp/Light, Part #MX2202; Onset Corporation, Bourne, Mass.) configured to collect light intensity and temperature data with a sampling frequency of one measurement every 15 minutes for the duration of the light exposure period. Data was extracted from the sensor using the HOBOmobile app (Onset Corporation) for analysis in Mircrosoft Excel. Data collected by sensor SN 20156615 showed the light intensity average of 18,330 lux (standard deviation of 3,288 lux) and temperature of 10.7 C (standard deviation of 1.6 C).

### Riboflavin Chemical Analysis of Milk Samples—Approach 1, Approach 2

Milk samples that were portioned and reserved for riboflavin analysis were kept in dark frozen storage until analysis. Riboflavin content of these samples was analyzed using a fluorometric method (AOAC method 970.65) (Webster et al., 2009) and measured on a Shimadzu RF-1501 Spectrofluorophotometer (Shimadzu Scientific Instrument, Inc., Columbia, Md., U.S.A.) as described by Wang et al. (2018). For each bottle condition for each simulated retail storage interval, the riboflavin content was measured in the milk as the average of the replications.

### Accelerated Retail (AR) Isothermal Light Exposure System and Riboflavin Chemical Analysis of Riboflavin Solutions—Approach 3

The same set of package conditions, two of each condition denoted −1 and −2 on their codes, were filled with RF solutions and placed into the accelerated retail isothermal exposure system. The packages were distributed on the two shelfs, denoted A and B, on the outer perimeter of a rotating carousal with a solid base. The package conditions were arranged such that one of each condition was present on each of the two shelves. For the first trial (replication #1), the replicate packages denoted −1 were placed on shelf A with those labeled −2 placed on shelf B. The packages switched shelf locations for the second trial (replication #2).

At defined intervals, the light exposure was briefly paused, the study packages were removed, uncapped, assed for RF content as described in Example 1, recapped and returned to the light exposure device to resume light exposure. This process took approximately 20 minutes and was repeated a light exposure duration intervals of 0, 60, 120, 240, 360, 1367, and 2756 minutes for replication #1 and 0, 63, 123, 183, 397, 1355, 1795, 2775, 3152, 4232, 4592 and 9992 minutes for replication #2. Concurrent with the light exposure, the temperature and light intensity were tracked within the chamber to ensure a uniform light exposure environment as described earlier.

Using the same equipment and approach as described earlier, data was collected by sensor SN 20656616 and showed a light intensity average of 12,104 lux (standard deviation of 1,185 lux) and a temperature of 8.1° C. (standard deviation of 0.3° C.). The light and temperature data were filtered to remove the changes in light intensity observed when the door, 2 FIGS. 1 and 2, was opened for measurement as described above. Additionally, the temperature data was filtered to remove the defrost cycle which is programed into the device control system to occur every 6.5 hours during which the temperature is moderately elevated from the control level for approximately 25 minutes.

### Result and Data Comparison of Approaches 1, 2, and 3

The three described approaches each yielded a data set of the RF concentration data as a function of the light exposure duration for each package condition. Using the data reduction approach illustrated in Example 1, the RF data were modeled for the pseudo first order reaction kinetics decay of RF as a function of package condition. It was found this model approximated the data well for RF degradation to 90% decline (In [RF] greater than −2 with [RF] in units of mg/mL) with correlations coefficients exceeding values of R²>0.80 for package conditions where the RF declined more than 5% from its initial value. From this model, the pseudo first order decay constants (k′) for RF were determined as presented in Table 16. Where RF decline was very low or not detected as seen in package conditions F and S, the value of k′ could not be determined. Package condition F was not studied in Approach 3.

A high degree of correlation between these rate constants data can be seen by plotting these data by package condition illustrating how the three approaches explored are correlated to one another. Thus, the light protection performance of a given package condition as part of a package system for protecting RF from the impacts of light can be readily assessed.

TABLE 16
Pseudo first order decay constants (k′) for RF as a
function of light exposure and package contents approach

| | k′ (1/day) | | |
|---|---|---|---|
| | Approach 1 | Approach 2 | Approach 3 |
| Package | SR light exposure | AR light exposure | AR light exposure |
| Condition | of milk | of milk | of RF solution |
| N | 1.11E−01 | 0.97 | 5.90E−04 |
| L | 4.04E−02 | 0.23 | 8.76E−05 |
| M | 9.86E−03 | 0.20 | 5.32E−05 |
| S | 1.00E−03 | ND¹ | 4.20E−06 |
| F | 5.71E−04 | ND¹ | NS² |

¹ND—no detectable decline;
²NS—not studied

It is clear that the accelerated light exposure device and methods of the application can be useful to rapidly assess a package system for its photoprotective performance and to predict the associated impact on light sensitive nutrients, such as demonstrated for RF in this example, contained in a package system as either as a solution or as a complete product under such light exposure conditions. The methods and devices shown in this example enable accelerated experimentation. While the SR light exposure conditions of Approach 1 required 26 weeks (182 days) of light exposure, the AR conditions that were used to provided light exposure to enable evaluation of the light protection performance of the packages containing the milk and riboflavin solutions in Approaches 2 and 3 were conducted much faster. As shown in Table 2, Approach 2 light exposure was completed in 16 days and Approach 3 light exposure was completed in 2 days.

The acceleration factor for the light exposure approaches of this example are shown in Table 17. The milk system under AR light exposure conditions of Approach 2 was monitored for 16 days of accelerated light exposure. This represents an acceleration factor for exposure time over the SR exposure of Approach 1 of 11.4 times. The RF solution system under AR light exposure conditions of Approach 3 was monitored for 3.2 days representing an acceleration factor for exposure time over the SR exposure of Approach 1 of 57.1 times.

The loss of RF in milk under the AR conditions was correlated to the SR conditions with a correlation coefficient of 0.93 with the linear fit shown in FIG. 22. The rate of degradation of RF in milk was 8.4 times faster by AR light exposure than SR light exposure.

Thus, these data demonstrate that the methods and devices provide a useful means to monitor the photoprotective performance of a package system. Further it is shown that the AR approach and device provide a benefit of faster package system exposure enabling rapid and quantitative evaluation of the photoprotective performance of a package system. While the results are accelerated, the excellent correlation (R²>0.9) to the effects observed under lighting like that often found in retail circumstances (SR) is demonstrated.

TABLE 17

| | | | | Light | Milk RF |
|---|---|---|---|---|---|
| | | | Light | Exposure | Decay |
| | Light | Package | Exposure | Acceleration | Acceleration |
| Approach | Exposure | Contents | Time (d) | Factor | Factor |
| 1 | SR | Milk | 182 | 1 | 1 |
| 2 | AR | Milk | 16 | 11.4 | 8.4 |
| 3 | AR | RF | 3.2 | 57.1 | NA |
| | | solution | | | |

### REFERENCES

Wang, A., Dadmun, C. H., Hand, R. M., O'Keefe, S. F., J'Nai, B. P., Anders, K. A., et al. (2018). Efficacy of light-protective additive packaging in protecting milk freshness in a retail dairy case with LED lighting at different light intensities. Food Research International, 114, 1-9.
Webster, J. B., Duncan, S. E., Marcy, J. E., & O'Keefe, S. F. (2009). Controlling light oxidation flavor in milk by blocking riboflavin excitation wavelengths by interference. Journal of Food Science, 74, 390-398.

1. A method for determining a photoprotective performance value of a package system, the method comprising:
(a) providing a light exposure chamber;
(b) providing a package system containing one or more samples to form a filled package system;
(c) providing at least one light source;
(d) equilibrating the filled package system to a desired control temperature;
(e) positioning the filled package system at a desired distance from the at least one light source;
(f) exposing the filled package system to the at least one light source for one or more durations while maintaining the desired control temperature;
(g) measuring changes to the sample at the one or more durations to generate one or more data points;
(h) using the one or more data points to determine the photoprotective performance value of the package system.

2. The method of claim 1, wherein the at least one light source is located a fixed distance from the filled package system.

3. The method of claim 2, wherein the filled package system is rotated about the at least one light source during step (e).

4. The method of claim 3, wherein the filled package system is rotated about its axis during step (e).

5. The method of claim 1, wherein the at least one light source rotates about the filled package system.

6. The method of claim 1, wherein the at least one light source traverses across a surface of the filled package system during step (e).

7. The method of claim 3, wherein the at least one light source rotates about its axis during steps (e) and (f).

8. The method of claim 2, wherein the at least one light source comprises at least two light sources.

9. The method of claim 1, wherein the sample is an unknown entity.

10. The method of claim 1, wherein the sample is a known entity.

11. The method of claim 1, wherein the sample comprises one or more photosensitivity entities.

12. The method of claim 11, wherein the one or more photosensitive entities comprise at least one material selected from the group consisting of riboflavin, natural and synthetic food additives, natural and synthetic dyes, and natural and synthetic pigments (e.g., curcumin, erythrosine); chlorophyll (all variants); myoglobin, oxymyoglobin, and other hemeproteins; water and fat soluble essential nutrients, minerals, and vitamins (e.g., riboflavin, vitamin A, vitamin D); food components; oils (e.g., soybean oil); proteins (e.g., proteins derived from the amino acids tryptophan, histidine, tyrosine, methionine, cysteine, etc.); pharmaceutical compounds; personal care and cosmetic formulation compounds and their components; household chemicals and their components; and agricultural chemicals and their components.

13. The method of claim 1, wherein the package system comprises a container and closure assembly.

14. The method of claim 1, wherein the package system comprises a container portion and a closure.

15. The method of claim 14, wherein the container portion has an average wall thickness of from about 2 mil to about 100 mil.

16. The method of claim 1, wherein the light source emits light from a point, line, or sphere and is selected from the group consisting of fluorescent lights, arc discharge lamps, LEDs (light emitting diodes), electromagnetic radiation source (LED, Halogen, fluorescent (bio, non-bio, etc.), Luminescent (bio, non-bio, etc.), incandescent, Arc (Xe, carbon, etc.), LASER, MASER, X-ray, Radio, Microwave, the sun, the moon, etc.), either as is or conditioned (filtered (low-pass, high-pass, bandpass, multi-bandpass, etc.), polarized (described via any Jones vector, coherency matrix, Poincare Sphere, etc.), amplified via gain medium, attenuated, etc.) either coherent or incoherent, and laser light sources.

17. The method of claim 1, wherein the desired control temperature is from about −1° C. to about 60° C.

18. The method of claim 1, wherein the one or more durations are from about one second to about 10,000 minutes.

19. The method of claim 3, wherein the filled package system comprises multiple filled package systems.

20. The method of claim 3, wherein the filled package system is contained within a secondary package.

21. The method of claim 1, wherein at least one of the filled package system and the at least one light source can be stationary or in motion during step (e).

22. The method of claim 21, wherein the at least one filled package system is in at least one of rotational motion, translational motion, and orbital motion during step (e).

23. The method of claim 21, wherein the at least one light source is in at least one of rotational motion, translational motion, and spherical motion during step (e).

24. The method of claim 1, wherein the at least one filled package system and the at least one light source are located within an isothermal system during step (e).

25. The method of claim 1, wherein the at least one filled package system comprises an inert atmosphere.
